(19) United States
(12) Patent Application Publication
(10) Pub. No.: US 2010/0144865 A1
(43) Pub. Date: Jun. 10, 2010

(54) COMPOUND FEED FOR AQUACULTURE

(76) Inventors: Jana Pickova, Uppsala (SE); Afaf Kamal-Eldin, Vittinge (SE); Sofia Trattner, Uppsala (SE); Eva Brännäs, Obbola (SE)

Correspondence Address: BIRCH STEWART KOLASCH & BIRCH, PO BOX 747, FALLS CHURCH, VA, 22040-0747, US

(21) Appl. No.: 12/598,756

(22) PCT Filed: Apr. 25, 2008
(86) PCT No.: PCT/SE2008/050477

§ 371 Date: Dec. 15, 2009

(30) Foreign Application Priority Data
May 4, 2007 (SE) 0701109-1

(51) Int. Cl. A61K 31/34; A61K 31/20; A61K 31/122; A01N 31/14

(52) U.S. Cl. 514/470; 514/558; 514/688; 514/689; 514/721

(57) ABSTRACT

The present invention relates to a fish feed comprising dioxabicyclo[3.3.0]octane derivates, in particular selected sesame lignans, and a vegetable oil or oil mixture comprising at least one 18-carbon chain omega-3 fatty acid. The dioxabicyclo[3.3.0]octane derivate will induce a desaturation and elongation of the 18-carbon chain fatty acid in the fish muscle into longer carbon chain polyunsaturated fatty acids.

### TECHNICAL FIELD

The present invention generally relates to a novel compound feed for aquaculture, and in particular to such a feed having capability of altering muscle fatty acid composition in species reared in aquaculture.

### BACKGROUND

Today, there is a great demand for natural pelagic fish populations as basis for oil of marine origin in feed products of aquaculture industry. This need has outstripped the current supply of oil manufactured from wild caught marine and fresh water species, and it is already common practice within the fish industry to replace fish oil with plant oils. In a longer perspective, the increasing aquaculture production makes it necessary to find new and land-based raw materials to, at least partly, replace the depleting fish sources in aquaculture fish feed.

In a longer perspective, the increasing aquaculture production makes it even more necessary to find new and land-based raw materials to, at least partly, replace the depleting sources of raw materials originating from fish and other species in fresh and marine waters in aquaculture feed products.

It is expected that new aquaculture diets will heavily rely on the use of alternative oil ingredients like, but not limited to, plant, bacteria, and/or algae derived oil ingredients. For example, in particular plant-derived oils are foreseen to become important ingredients in compound feed for aquaculture also for carnivorous, cold and temperate water aquaculture species which are the most farmed aquaculture species. However, certain components of plant oils may induce xenobiotic effects on these aquaculture species. Therefore, the replacement of fatty acids originating from oils from fresh and marine species with plant sources in compound feed for aquaculture demands new insights that take into account the beneficial as well as the adverse effects of plant-derived phytochemicals. Compound feed for aquaculture including plant oils differ in their biochemical composition compared to feeds based on oils from species living in fresh and marine waters. So far, studies have mostly concentrated on the production yield when aquaculture species like fish are grown on feeds with a partial replacement of oils originating from fresh water and marine species by plant oils, e.g. linseed, hempseed, soybean and rapeseed oils. It has been shown that up to 50% replacement of oils originating from fresh water and marine species by several plant oils does not alter the growth of aquaculture species and feeds with a replacement of 10 to 25% of the plant oil with oil originating from fresh water and marine species are frequently commercialized.

A major drawback of the prior art of plant oil-based feeds for aquaculture species is the decreased amount of eicosapentaenoic acid (EPA), docosapentaenoic acid (DPA), docosahexaenoic acid (DHA) and to some extent arachidonic acid (AA) and an increase in n-6 fatty acids, especially linoleic acid (LA). In the muscle of aquaculture species, not only the fatty acid composition will be altered towards a lower amount of “marine” fatty acids, notably DHA and EPA, but also the content of several bioactive compounds including phytoestrogens, phytosterols, and other phenolic compounds, may be increased.

In herbivorous and omnivorous warm water aquaculture species, experiments with different plant ingredients have been performed more extensively as exemplified by the documents [1-3] presented below. Feeding regimens containing plant oils are, though, not exotic to these aquaculture species as they are in line with their natural food chain.

Document [1] discloses the effects of different supplementary feeds on the growth rate of Indian major carp Catla catla. Nine different supplementary feeds made from locally available ingredients in India were manufactured and used as supplements to natural feed. The best results in terms of gain in total weight, total length and body depths of fingerlings of Catla catla were obtained with oiled and/or deoiled rice bran. Sesame cake mixed with oiled or deoiled rice bran were also tested as supplementary feed but resulted in a decrease of total body depth (thickness) as compared to control feed.

Document [2] discloses usage of different supplementary feeds in three experimental sets with the supplementary feeds alone or complemented with frozen zooplankton or live zooplankton for Indian major carp Catla catla. In all experiments, reduction in total length, total body depth and total weight were observed for all supplementary feeds. One of the feeds contained a mixture of sesame cake and rice bran.

Document [3] discloses the usage of different supplementary fish feeds made from locally available ingredients in Bangladesh for a carp polyculture system (40% Catla catla, 20% Labeo rohita, 20% Cirrhinus mrigala, 5% Cyprinus caripo and 15% Puntius gonionotus). One of the fish feed contained fish meal, wheat bran, sesame cake, molasses and a pre-mixture of vitamins and minerals. The feed that contained rice bran and mustard oil cake instead of wheat bran and sesame cake resulted in highest gross production of fish, highest carcass protein, protein efficiency ratio and apparent net protein utilization and best apparent feed conversion ratio. This feed also resulted in the highest benefit-to-cost ratio (BCR), whereas the feed containing wheat bran and sesame cake had the lowest BCR.

### SUMMARY

The present invention overcomes drawbacks of the prior art feeds based on vegetable oils, including the decrease in highly unsaturated fatty acids (HUFA).

It is a general object of the present invention to provide an aquaculture feed altering fatty acid composition in aquaculture species fed with the feed.

It is another object of the invention to provide an aquaculture feed increasing the relative content of omega-3 fatty HUFA in the aquaculture species.

These and other objects are met by the invention as defined by the accompanying patent claims.

Briefly, the present invention involves a compound feed for aquaculture species comprising i) a lignan represented by formula (I):

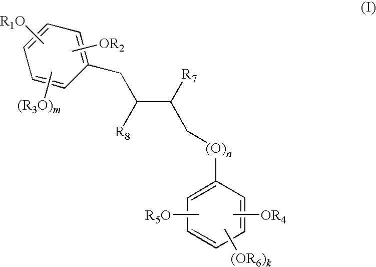

wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, m and n each independently represents 0 or 1, and R₇ and R₈ independently represents a methyl group or —CH₂—O— forming a tetrahydrofuran group with the lignan backbone, ii) an oil or oil mixture containing a 18-carbon chain omega-3 fatty acid, and iii) an antioxidant.

The lignan of the present invention is preferably a dioxabicyclo[3.3.0]octane derivate represented by formula (II):

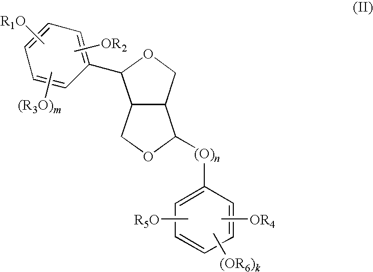

The dioxabicyclo[3.3.0]octane derivates of the present invention are preferably sesame lignans, including sesamin, episesamin and/or sesamolin. The derivates can be present in a pure form in the feed or in the form of different natural or processed sesame products comprising the lignans, such as sesame seeds, sesame oil, sesame meal or sesame cake.

The oil can be a single plant or vegetable oil comprising the omega-3 fatty acid or a mixture of at least two plant oils, of which at least one comprises the omega-3. The omega-3 fatty acid of the oil is preferably α-linolenic acid and/or stearidonic acid. In addition, the oil or oil mixture can also contain an 18-carbon chain omega-6 fatty acid, such as linoleic acid.

The preferred antioxidant is employed for inhibiting oxidization of the high content of polyunsaturated fatty acids in the oil or oil mixture, thereby increasing the shelf life of the aquaculture feed. In addition, the antioxidant can, depending on which antioxidant or antioxidant mixture that is employed, result in a coloring of the species fed on the compound feed for aquaculture based on this invention.

The lignans of the present invention will cause an elongation and desaturation of the 18-carbon chain omega-3 and omega-6 fatty acid in the aquaculture species, in particular in the triacylglycerols and phospholipids of white muscle. As a consequence, these shorter carbon chain omega-3 and omega-6 fatty acids are transformed into longer carbon chain omega-3 and omega-6 HUFA, including eicosapentaenoic acid (EPA), docosapentaenoic acid (DPA), docosahexaenoic acid (DHA) and arachidonic acid (AA).

A particular embodiment of the compound feed for aquaculture comprises the lignan defined by formula (I), an antioxidant and an oil or oil mixture having a total 18-carbon chain omega-3 fatty acid content of at least 5% by weight of the oil or oil mixture, preferably at least 10%, such as at least 20% and more preferably at least 30% or even about or at least 40% by weight or sometimes even more.

Another particular embodiment of the aquaculture feed comprises the sesame lignan 2,6-bis-(3,4-methylenedioxy phenyl)-trans-3,7-dioxabicyclo[3.3.0]octane, an antioxidant and an oil or oil mixture comprising an 18-carbon chain omega-3 fatty acid. The feed preferably also comprises at least one additional sesame lignan, preferably sesamin and/or sesamolin.

The compound feed for aquaculture of the present invention will, through the induced fatty acid elongation and desaturation, change the fatty acid composition of the muscles and other edible organs and tissues of the aquaculture species. As a consequence, this changed fatty acid composition will be closer to the fatty acid composition of corresponding wild or farmed species fed compound feed for aquaculture based on oils or meals from wild living species compared to the prior art of plant oil-based compound feed for aquacultures. The invention therefore provides an improved marine fatty acid profile and quality both for the welfare of the aquaculture species and as aquaculture species are consumed by humans or given as animal food.

The invention offers the following advantages:
Increases the proportion of omega-3 HUFA in aquaculture species fed the innovative compound feed for aquaculture;
Affects the fatty acid composition in the muscle and other edible organs of aquaculture species towards fatty acid compositions in wild species and farmed aquaculture species fed compound feeds based on oils/meals from wild caught species;
Leads to a healthier fatty acid composition in the muscles and other edible organs of aquaculture species when consumed as human food;
Allows production of plant oil-based compound feed for aquaculture as a replacement or complement to aquaculture compound feeds traditionally based on oils and meals from fresh and marine water species included in aquaculture compound feed for aquaculture species; and
Sesame accumulates in aquaculture animals' muscles and can be used as a traceable biomarker and additionally has positive health effects in humans eating the animals.

Other advantages offered by the present invention will be appreciated upon reading of the below description of the embodiments of the invention.

### SHORT DESCRIPTION OF THE DRAWINGS

The invention together with further objects and advantages thereof may best be understood by making reference to the following description taken together with the accompanying drawings, in which:

FIG. 1 is a diagram illustrating the effect of feed components on the relative proportions of omega-3 fatty acids expressed as percentage of total fatty acid content in white muscle triacylglycerols;

FIG. 2 is a diagram illustrating the effect of feed components on the changes of α-linolenic acid and docosahexaenoic acid in white muscle triacylglycerol;

FIG. 3 is a diagram illustrating the effect of feed components on the desaturation index in white muscle triacylglycerol;

FIG. 4 is a diagram illustrating percentage distribution of oxaloacetate and matate (ox-mal) and acetate in the acid soluble fraction from 18:3 (n-3) oxidation;

FIGS. 5A to 5E illustrate relative expression of SRB, cd36, PPARα,β,γ in different sesamin concentrations, with significant differences to control group without sesamin denoted with *;

FIG. 6 illustrates relative expression of some lipid related genes in salmon, * indicates significant difference between control group and sesamin group (p<0.05); and

FIGS. 7A and 7B illustrate relative expression of Δ-5 and Δ-6 desaturase with significant differences between the groups denoted with *.

### DETAILED DESCRIPTION

The present invention generally relates to a new compound feed for aquaculture, the manufacture and use thereof. The aquaculture feed of the present invention is based on an oil or oil mixture, in particular a plant oil or oil mixture, complemented with one or more specific lignans, in particular dioxabicyclo[3.3.0]octane derivates or derivatives and sesame lignans. These lignans have the unexpected effect of altering the fatty acid composition in aquaculture species fed the feed. The aquaculture species will then have a fatty acid composition that is closer to the fatty acid composition in farmed aquaculture species fed a feed based on traditional ingredients from species living in fresh or marine waters or corresponding wild species. As a consequence, it is expected that aquaculture species fed a feed of the present invention will have a better fatty acid composition when used as a food source for humans and other animals as compared to aquaculture species fed prior art plant oil-based feeds.

The compound feed for aquaculture of the present invention can be used as a primary feed for the aquaculture species or as a supplementary feed, complementing a traditional feed based on raw materials originating from species living in fresh and marine waters.

The feed of the present invention can be generally be used as feed for any aquaculture species. However, the feed is in particular adapted for carnivorous aquaculture species and preferably carnivorous cold or temperate water aquaculture species. Examples of such species include aquaculture species farmed on the Northern and Southern hemispheres, especially in Europe, the Americas, Japan, China and Australia. These species include aquaculture species belonging to the family Salmonidae, the orders Gadiformes and Perciformes and flat fishes. Examples of Salmonidae species include species of the genus Coregonous (whitefishes), Prospoium (round whitefishes), Stenodus (inconnus), Thymallus (graylings), Brachymystax (lenoks), Hucho, Oncorhynchus (pacific salmon and trout), Parachucho, Salmo (Atlantic salmon and trout), Salvelinus (char and trout) and Salvethymus. Gadiformes is the order of ray-finned fish and include the family of Gadidae, which comprises, among others, cod, haddock, whiting, hake, seith and Pollock. Perciformes include both freshwater and saltwater species, e.g. species of the Percidae family, such as perch pike-perch, species of the Sparidae family, such as sea breams, species of the Serranidae family, such as sea bass, and species of the Scombridae, such as Southern Bluefin Tuna (Thynnus maccoyii). Other perciformes include Yellowtail (Seriola sp.), Wolffish (Anharhichas lupus) and Barramundi (Lates calcarifer). In addition, different flat fish species (of several different families) can benefit from the feed of the invention, e.g. turbot (Psetta maxima, Scopthalmus rhombus), soles (Soleace), flounders, plaice and Atlantic halibut. Also Pleuronectiformes, families Pleuronectidae, Scophthalmidae, Soleidae can be fed by the feed of the invention. The feed of the invention is though not limited to carnivorous aquaculture species and fishes but can also be given to non-carnivorous species, including carnivorous and non-carnivorous species of, for instance the Teleostei infraclass. A particular preferred aquaculture species of the invention is different fish species. Other preferred aquaculture species include telestes, crustaceans and mollusks

The aquaculture feed of the invention comprises a lignan represented by formula (I):

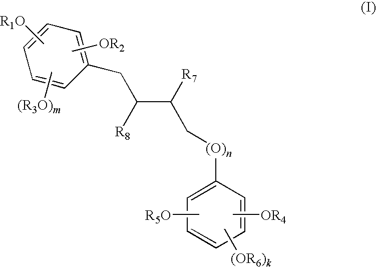

wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, m and n each independently represents 0 or 1, and R₇ and R₈ independently represents a methyl group or —CH₂—O— forming a tetrahydrofuran group with the lignan backbone.

Examples of an alkyl group having 1 to 3 carbon atoms include a methyl group, ethyl group, n-propyl group and isopropyl group. Examples of a sugar moiety include a monoglycoside, a diglycoside and a triglycoside moiety, such as mono-, di- or triglucoside, -galactoside, -fructoside or -glucuronide.

In a preferred embodiment of the invention the lignan is a dioxabicyclo[3.3.0]octane derivate represented by formula (II):

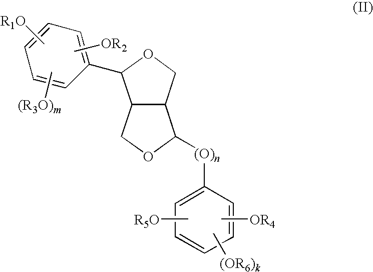

wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, m and n each independently represents 0 or 1. Thus, the octane derivate is R₇ and R₈ in the form of —CH₂—O— forming a respective tetrahydrofuran group with the lignan backbone to the structure II.

In a particular embodiment of the invention, R₁ and R₂ together represent a methylene group and R₄ and R₅ together represent a methylene group or R₄ is a hydrogen atom and R₅ is a methyl group. Preferably, k, m, n are all zero or k and m are zero and n is one.

The feed can comprise one of the lignans defined by formula (I), such as one of the dioxabiocyclo[3.3.0]octane derivates defined by the formula (II), or a mixture of at least two such lignans. This lignan mixture can be in any range from 1:0, i.e. only a first lignan, to 0:1, i.e. only a second lignan. The same principles apply mutatis mutandis to a lignan mixture of more than two lignan. In a particular example, an equimixture 1:1 of two lignans of the invention, such as sesamin and episesamin, can be used in the feed. In other particular examples more than two lignans of the invention are used in a mixture. In particular, different mixtures of sesamin and sesamolin as present in natural sesame oil can be used.

In a preferred embodiment, the lignan is a sesame seed lignan, or a derivate of such a sesame lignan. Specific examples of such lignans encompassed by the formula (II) include 2,6-bis-(3,4-methylenedioxy phenyl)-cis-3,7-dioxabicyclo[3.3.0]octane (sesamin); 2,6-bis-(3,4-methylenedioxy phenyl)-trans-3,7-dioxabicyclo[3.3.0]octane (episesamin); 2-(3,4-methylenedioxy-6-hydroxy phenyl)-6-(3,4-methylenedioxy phenyl)-cis-3,7-dioxabicyclo[3.3.0]octane (sesaminol); 2-(3,4-methylenedioxy-6-hydroxy phenyl)-6-(3,4-methylenedioxy phenyl)-trans-3,7-dioxabicyclo[3.3.0]octane (episesaminol); 2-(3,4-methylenedioxy phenoxy)-6-(3,4-methylenedioxy phenyl)-cis-3,7-dioxabicyclo[3.3.0]octane (sesamolin); 2-(3-methoxy-4-hydroxy phenoxy)-6-(3,4-methylenedioxy phenyl)-cis-3,7-dioxabicyclo[3.3.0]octane (sesamolinol); 2-(3-methoxy-4-hydroxy phenyl)-6-(3,4-methylenedioxy phenyl)-cis-3,7-dioxabicyclo[3.3.0]octane; 2,6-bis-(3-methoxy-4-hydroxy phenyl)-cis-3,7-dioxabicyclo[3.3.0]octane (pinoresinol); 2-(3,4-methylenedioxy-6-methoxy phenyl)-6-(3,4-methylenedioxy phenyl)-cis-3,7-dioxabicyclo[3.3.0]octane (sesangolin); and 2-(3,4-5-methylenedioxy phenyl)-6-(3,4-methylenedioxy-5-methoxy phenyl)-trans-3,7-dioxabicyclo[3.3.0]octane (2-episesalatin). Some of these compounds may be in the form of glycosides, and optically active substances and isomers are also included in the invention. Such glycosides include mono-, di- and triglycosides, especially of sesaminol, episesaminol, sesamolinol, pinoresionol or 2-(3-methoxy-4-hydroxy phenyl)-6-(3,4-methylenedioxy phenyl)-cis-3,7-dioxabicyclo[3.3.0]octane.

Preferred lignans of the present invention are sesamin, episesamin, sesamolin, a mixture of sesamin and episesamin, a mixture of sesamin and sesamolin, a mixture of episesamin and sesamolin, or a mixture of sesamin or sesamolin and at least one additional sesame lignan presented above.

Sesamin and sesamolin are the major oil-soluble lignans of sesame seeds. However, additional amounts of sesaminol, 2-(3-methoxy-4-hydroxy phenyl)-6-(3,4-methylenedioxy phenyl)-cis-3,7-dioxabicyclo[3.3.0]octane, sesamolinol and pinoresinol are also found in the sesame seeds in free or glucosilated forms. Moreover, the presence of other lignans has been reported in wild species of genus Sesamum, such as sesangolin in Sesamum angolense and Sesamum angustifolium and 2-episealatin in Sesamum alatum.

The lignans of the present invention can be obtained by extraction and concentration from sesame seed or oil using various organic solvents that are able to extract and dissolve the derivates. Such solvents include super-critical carbon dioxide, acetone, methyl ethyl ketone, diethyl ketone, methanol, ethanol or mixtures of these solvents. A particular example of such extraction involves uniformly mixing sesame oil with any of the above-mentioned solvents, allowing the mixture to stand undisturbed at a low temperature, performing phase separation in accordance with routine methods, such as centrifugal separation, and distilling off the solvent from the solvent fraction. More specifically, after dissolving sesame oil in 2 to 10 volumes, and preferably 6 to 8 volumes, of acetone, the solution is allowed to stand undisturbed overnight at −80° C. The resulting oil component forms a precipitate and the acetone is removed from the filtrate obtained by filtration to obtain an extract having as its main ingredient the lignan of the present invention. Alternatively, super-critical extraction and fractionation methods can be used.

In addition, another method for obtaining a product containing the lignans of the present invention comprises mixing sesame oil with hot methanol or hot ethanol, allowing the mixture to stand undisturbed at room temperature and distilling off the solvent from the solvent fraction. More specifically, after vigorously mixing sesame oil with 2 to 10 volumes and preferably 5 to 7 volumes of hot methanol or ethanol (50° C. or higher), phase separation is carried out in accordance with routine methods, such as by standing the mixture undisturbed at room temperature or by centrifugal separation. The solvent is distilled off from the solvent fraction to obtain an extract having for its main ingredient the derivative of the present invention. Moreover, the extract can also be obtained by utilizing super-critical gas extraction. The sesame oil to be used may be a finished product or any of the crude products in the production process of sesame oil prior to the decoloring step.

In addition, a product containing the lignan of the present invention can be obtained by extraction from sesame seeds or the defatted products sesame seeds (residual oil content: 8 to 10%). After crushing the sesame seeds or defatted products as necessary, extraction can be performed in accordance with routine methods by pressing or by using any of the above-given solvents. After separating the extraction residue, the solvent is removed from the extract liquid by evaporation and so forth to obtain the extract.

Further purification can be performed using traditional methods such as column chromatography, high-performance liquid chromatography, recrystallization, distillation and liquid-liquid exchange distribution chromatography. More specifically, after fractionating the above-mentioned extract with high-performance liquid chromatography using a reversed phase column and methanol/water for the eluent, and distilling off the solvent, the resulting crystals are recrystallized with ethanol to obtain the lignan.

In addition, the lignan of the present invention can also be obtained by synthesis in accordance with routine methods. Episesamin is a sesame lignan that is not naturally occurring in natural sesame products. It is, however, produced during refining of sesame oil and is variably present in refined sesame oils. It can thus be manufactured from sesamin, for instance, by bleaching in the presence of acidic clay. For more information of the production of sesame lignans that can be used according to the present invention reference is made to the document [4].

Further examples of procedures of obtaining the lignans of the invention include liquid-liquid countercurrent distribution chromatography and acetone preparation of TAG. These techniques are well-known in the art and are not further described herein.

There are several other sources of the lignans of the present invention in addition to sesame seeds. For instance, lignans of the invention can be extracted different tree species, such as from the heart wood and/or leaves from, for instance, Hinoki cypress (Chamaecyparis obtuse), Chamaecyparis pisiferia, Chamaecyparis obtusa, Chamaecyparis obtusa cv. Breviramea, Chamaecyparis pisifera cv. Plumosa-aurea, Chamaecyparis lawsoniana, Paulownia species. Also other plant sources, including tea seeds (Camellia oleifera), can be used as lignan source.

The lignans of the present invention must not necessarily be used in highly pure form in the feed. Thus, substances that contain the lignans of the invention and which may be obtained from naturally occurring sources can be used according to the present invention. These include sesame seeds, sesame oil, such as crude/virgin, roasted or refined oil, and sesame cakes or similar products from different species of the genus Sesamum. A preferred derivate source of the present invention is sesame cake comprising a mixture of sesame lignans having the fatty acid elongation and desaturation effect in fed aquaculture species.

The compound feed for aquaculture feed of the invention also comprises an oil or a mixture of oils, preferably plant oils. In addition, the oil or oil mixture comprises an 18-carbon chain omega-3 fatty acid and optionally an 18-carbon chain omega-6 fatty acid. These 18-carbon chain fatty acids will, when digested by the aquaculture species, be elongated and desaturated into longer chain fatty acids due to the presence of the lignan of the present invention. Thus, the inclusion of the lignan will trigger a conversion of these shorter chain omega-3 and omega-6 fatty acids to the corresponding longer chain omega-3 and omega-6 fatty acids in the aquaculture species. This causes an increase in the amounts of eicosapentaenoic acid (EPA, 20:5 (n-3), eicosa-5,8,11,14,17-pentaenoic acid), docosapentaenoic acid (DPA, 22:5 (n-3), docosa-7,10,13,16,19-pentaenoic acid), docosahexaenoic acid (DHA, 22:6 (n-3), docosa-4,7,10,13,16,19-hexaenoic acid) and arachidonic acid (AA, 20:4 (n-6), 5,8,11,14-eicosatetraenoic acid), while the amounts of α-linolenic acid (ALA, 18:3 (n-3), octadeca-9,12,15-trienoic acid) and linoleic acid (LA, 18:2 (n-6), 9,12-octadecadienoic acid) are decreased.

As is well known in the art, a fatty acid can be denoted by Y:Z, where Y specifies the number of carbons in the carbon chain while Z specifies the number of double bounds in the carbon chain. If the fatty acid is an omega-3 fatty acid, the postfix (n-3) is used. An omega-6 fatty acid correspondingly has (n-6) as postfix.

The change in fatty acid composition brought by the feed of the present invention is particularly taking place in white muscles of the aquaculture species, while fatty acids in red muscle and liver were only slightly affected. The white muscles represent the main muscle part in the most important aquaculture species, approximately 95%, so the change in fatty acid composition caused by the present invention will have a major impact to the total fatty acid composition in the aquaculture species. The lack of effect of the lignan of the invention in modifying the fatty acid composition in liver may be positive for the welfare of the aquaculture species, as the species might otherwise experience negative effects on the liver function. The change in fatty acid composition in the white muscles was detected both in the phospholipid fraction (PL) and in the triacylglycerol fraction (TAG).

Without being bound by theory, it is expected that the lignans of the present invention affect the genes involved in the lipid metabolism in the aquaculture species, in particular in white muscles. This gene activation may lead to a modulation of the activity of the Δ5 desaturase and/or Δ6 desaturase activity, which two enzymes are involved in the elongation and desaturation of 18:3 (n-3) and 18:4 (n-3) into 20:4 (n-3), 20:5 (n-3), 22:5 (n-3) and 22:6 (n-3) and elongation and desaturation of 18:2 (n-6) into 20:4 (n-6).

Suitable oils that can be used according to the present invention include plant oils having a high 18-carbon chain omega-3 content. Thus, the oil or oil mixture preferably has a total 18-carbon chain omega-3 content of at least 5% by weight of the oil or oil mixture, preferably at least 10% by weight and more preferably at least 20%, such as at least 30% by weight or about or at least 40% by weight. The oil or oil mixture of the present invention preferably also has a high polyunsaturated fatty acid content. Preferably this polyunsaturated fatty acid content is at least 40% and more preferably at least 50% of the total fatty acid content of the oil or oil mixture.

Preferred oils can be selected from the group of linseed/flaxseed oil, perilla seed oil, soybean oil, rapeseed oil, hempseed oil, black current seed oil and oils from the Boraginaceae family. The compound feed for aquaculture can contain a single oil or a mixture of at least two of the above-mentioned oils or other similar oils. As some of these oils have a very high 18-carbon chain omega-3 content, it is also possible to provide an oil mixture of at least one of the above-mentioned oils and another plant oil with low or no 18-carbon chain omega-3 or 18-carbon chain omega-6 content, such as sunflower oil or sesame oil.

The oil is preferably present in at least 10% by weight of the feed, more preferably at least 20% by weight, such as 20 to 35% by weight. Correspondingly, the lignan of the invention is preferably present in at least 0.5% by weight of the oil or oil mixture in the feed, more preferably at least 1% by weight, such as at least 2% by weight of the oil or oil mixture.

The aquaculture feed of the present invention furthermore preferably comprises an antioxidant or a mixture of antioxidants. The antioxidative effect is very advantageous for the shelf life and integrity of the high oil content of the feed. Thus, the antioxidants protect the polyunsaturated fatty acids in the feed from oxidation during storage. These polyunsaturated fatty acids are otherwise readily oxidized in air because of their unsaturated bonds. If such an oxidation is taking place at a great extent, the amount of 18-carbon chain omega-3 fatty acids in the feed will diminish, leading to a less positive effect for animals feeding on the feed of the invention.

Preferred antioxidants of the invention preferably have advantageous health effects to the aquaculture species feeding on the feed of the invention. Such antioxidants include carotenoids, vitamin E, tocopherols, tocotienols.

Carotenoids are organic pigments that are naturally occurring in chromoplasts of plants and some other photosynthetic organisms like algae, some fungus and bacteria or can be manufactured synthetically. The carotenoids of the present have dual functions in the feed. Firstly, they are antioxidants. Secondly, the carotenoids of the invention can also affect the coloring of the aquaculture species feeding on the carotenoid-containing feed. For instance, the pink color of salmon is due to the presence of carotenoids in the salmon feed. This means that the carotenoids contribute to a more inviting and enticing appearance of the species when used as human food.

Astaxanthin is an example of a preferred carotenoid that can be used in the feed of the invention. Astaxanthin is a xanthophyll that causes a coloring of Salmonidae fishes. Cantaxanthin is another carotenoid example that can be used according to the present invention. Cantaxanthin is traditionally used in similar applications as astaxanthin and the two carotenoids may also be used in combination. If the feed of the invention is intended for Salmonidae species, the antioxidant is preferably astaxanthin or a mixture of astaxanthin and at least one other antioxidant.

Preferred tocopherols and tocotrienols include α-, β-, γ-, δ-tocopherols and tocotrienols.

The antioxidant or mixture of at least two antioxidants is preferably added to the aquaculture feed to have a final concentration traditionally employed in aquaculture feeds. In a typical practical application, the feed can comprise at least 5 mg antioxidant per kilogram dry weight of the feed. In a preferred embodiment, the antioxidant concentration is at least 10 mg/kg, preferably at least 20 mg/kg, such as at least 40 mg/kg, at least 50 mg/kg or more preferably 60 mg/kg by dry weight of the feed.

The feed of the present invention may contain one or more additional ingredients besides the oil (mixture), the antioxidant and the lignan. These ingredients can be selected from traditional additives and ingredients of the prior art (supplementary) aquaculture feeds. Non-limiting examples include casein, gelatin, meal processed from fresh water or marine water species, corn, wheat gluten, starch and other cereal products, soy lecithin and other soy products, dextrin, vitamins, minerals, Ca₃SO₄, sodium alginate, etc. In such a case, the concentration of the lignan of the present invention may preferably be in the range of 0.1 to 10% by dry weight of the feed.

The main constituents of the present invention, i.e. the lignan and the oil or oil mixture, can be manufactured separately and even be provided as separate pellets. A compound feed of the invention may then be provided by mixing the two main constituents before or in connection with the feeding of the actual aquaculture species. It is also possible to administer the two constituents without prior mixing, i.e. feeding the species sequentially or substantially simultaneously with the derivate and the oil.

The final compound feed for aquaculture can for instance be in the form of pellets, powder or in a wet form.

A first aspect of the invention relates to a compound feed for aquaculture comprising a lignan represented by formula (I), an oil or oil mixture having a total 18-carbon chain omega-3 fatty acid content of at least 5% by weight of the oil or oil mixture and optionally but preferably an antioxidant. The feed may optionally include well-known aquaculture feed additives.

A second aspect of the invention defines a method of manufacturing the compound feed for aquaculture of the first aspect. This method involves mixing the lignan(s) with the oil or oil mixture and the antioxidant(s), optionally also involving adding well-known feed additives. If including more than two ingredients, the feed can be manufactured by mixing them in any order. The ingredients may be ground prior to or after mixture and can then be pressed into feed pellets of selected sizes, feed powder or in some other form.

A third aspect of the invention relates to the use of the lignan represented by formula (I) as an additive to an oil-containing aquaculture feed having a 18-carbon chain omega-3 fatty acid content of at least 5% by weight of the oil and comprising at least one antioxidant.

A fourth aspect of the invention relates to a compound feed for aquaculture comprising episesamin, an antioxidant and an oil or oil mixture comprising an 18-carbon chain omega-3 fatty acid. The feed preferably also comprises sesamin and may optionally include well-known aquaculture feed additives.

A fifth aspect of the invention defines a method of manufacturing the aquaculture feed of the fourth aspect. This method involves mixing the episesamin with the oil or oil mixture and the antioxidant, optionally also involving adding sesamin and/or well-known aquaculture feed additives. If including more than two ingredients, the feed can be manufactured by mixing them in any order. The ingredients may be ground prior or after mixture and can then be pressed into feed pellets of selected sizes.

A sixth aspect of the invention relates to the use of episesamin as an additive in an oil-based, antioxidant-comprising aquaculture feed, preferably such a feed comprising one or more 18-carbon chain omega-3 fatty acids.

The feeds of the invention can be used for altering the muscle fatty acid composition in an aquaculture species. This composition altering involves feeding the species with the feed to thereby induce the elongation and desaturation of the 18-carbon chain omega-3 fatty acids in the feed into longer chain polyunsaturated omega-3 fatty acids, preferably into such omega-3 fatty acids having 20 or 22 carbon atoms in their respective carbon chains.

A seventh aspect of the invention relates to the use of the lignan represented by the formula (I) in increasing a proportion of N-carbon chain omega-3 fatty acids in muscle of the aquaculture species, where N is larger than 18. In a preferred embodiment, N is 20 or 22.

An eighth aspect of the invention relates to the use of the lignan represented by the formula (I) in increasing elongation and desaturation of 18:3 (n-3) and/or 18:4 (n-3) in muscle of the aquaculture species.

The feed of the present invention has a further advantageous effect if the lignin is sesamin or a mixture of sesamin and at least one additional lignan. Sesamin present in the feed is partly taken up by the aquaculture species and becomes accumulated in the muscles. Sesamin can therefore be used as a traceable marker for detecting the presence of sesamin in the feed of the aquaculture species.

Furthermore, if the aquaculture species are then used as animal or human food, the sesamin in the muscle of aquaculture species will have several beneficial effects. Today sesamin is used as strong antioxidant. It has also shown great promise as a lipid oxidizer as well as to support anti-inflammatory actions. Its main action mechanism for fatty acid oxidation (fat burning) is triggered by peroxisome proliferator activator receptor alpha (PPAR-α). Sesamin-containing products are currently sold in sports nutrition and health food stores.

In a particular embodiment of the present invention, the lignan of the feed is exchanged for a 1,3-benzodioxole or a 1,3-benzodioxole derivate. 1,3-benzodioxole is represented by formula (III):

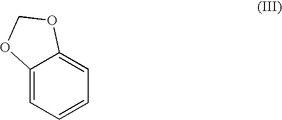

A 1,3-benzodioxole derivate is defined as a chemical substance having a functional 1,3-benzodioxole moiety connected to at least one side chain X connected to the benzodioxole moiety as defined by formula (IV):

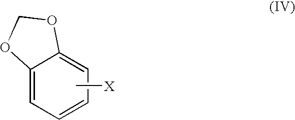

In such a case, depending on the side chain X, the derivate could be sesamin, episesamin, sesamolin, episesaminol, sesaminol, piperitol, sesamolinol, episesaminone, sesangolin, 2-episesalatin. Other examples of 1,3-benzodioxole derivates include 5-[1-[2-(2-ethoxyethoxy)ethoxy)ethoxy]-1,3-benzodioxole (sesamex) illustrated by formula (V):

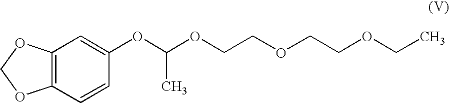

A further example is piperonyl butoxide (PBO), also known as 5-[2-(2-butoxyethoxy)ethoxymethyl]-6-propyl]-1,3-benzodioxole having formula (VI):

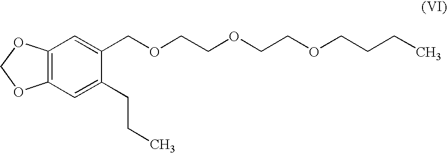

In this aspect, the aquaculture feed comprises 1,3-benzodioxole or a 1,3-benzodioxole derivate, an oil or oil mixture having a total 18-carbon chain omega-3 fatty acid content of at least 5% by weight of the oil or oil mixture and optionally but preferably an antioxidant. The feed according to this aspect can be used in the same way as the previously discussed feeds.

### EXPERIMENTS

The effect of sesamin lignans on fatty acid compositions of rainbow trout (Oncorhynchus mykiss) was investigated. The results indicated that the lignans added to compound feed for aquaculture increased the percentage of DHA in the rainbow trout white muscle.

### Chemicals and Reagents

Sesame lignans were used in the form of a sesamin/episesamin mixture (1:1, w/w). Fatty acid peaks were identified by comparison with the standard mixture GLC-68 A (Nu-check Prep, Inc, Elysian, Minn., USA). All solvents and other chemicals were also purchase from Merck and were used without further purification.

### Animals and Diets

Rainbow trout was fed five different diets. Four groups were fed experimental diets based of plant oils, while one group was being fed a commercial fish feed based on fish ingredients (FO). The diets were:

1) fish oil diet (FO);
2) linseed oil diet (LO);
3) linseed oil diet with 2% (of fat content) sesame lignans (LO+S);
4) mixed oil (linseed oil:sunflower oil, 6:4) diet (MO); and
5) mixed oil diet with 2% (of fat content) sesame lignans (MO+S).

The feed was prepared according to the method of document [5]. The composition of the diet is shown in Table I and fatty acid composition of diets is shown in Table II.

TABLE I
composition of basic feed

| Component | Weight percentage |
|---|---|
| Casein | 18.6 |
| Gelatin | 3.1 |
| Fish meal | 21.8 |
| Dextrin | 9.8 |
| Lipid* | 21.8 |
| Vitamins + minerals^{⊥} | 0.3 |
| Ca₃SO₄ | 4.0 |
| Starch | 15.0 |
| Sodium alginate | 4.0 |

*Lipid source was either linseed oil or linseed oil:sunflower oil (6:4)
^{⊥}Includes astaxanthin and vitamin E

TABLE II
fatty acid composition of feed

| Fatty acids | FO | MO | MO + S | LO | LO + S |
|---|---|---|---|---|---|
| 14:0 | 7.5 | 0.2 | 0.2 | 0.2 | 0.3 |
| 16:0 | 15.0 | 6.0 | 6.1 | 6.0 | 6.1 |
| 16:1 | 3.3 | 0.2 | 0.2 | 0.2 | 0.3 |
| 18:0 | 3.1 | 3.6 | 3.6 | 3.5 | 3.4 |
| 18:1 (n-9) | 9.9 | 20.6 | 20.4 | 16.6 | 16.4 |
| 18:1 (n-7) | 2.6 | 0.6 | 0.6 | 0.6 | 0.6 |
| 18:2 (n-6) | 4.5 | 33.2 | 31.9 | 15.0 | 14.7 |
| 18:3 (n-3) | 1.2 | 31.7 | 32.4 | 53.4 | 52.5 |
| 20:0 | 1.1 | 0.2 | 0.2 | 0.1 | 0.1 |
| 20:1 | 5.6 | 0.1 | 0.1 | 0.05 | 0.1 |
| 22:0 | 0.5 | 0.4 | 0.3 | 0.1 | 0.1 |
| 22:1 | 0.9 | 0.2 | 0.2 | 0.1 | 0.2 |
| 20:5 (n-3) | 10.4 | 0.2 | 0.2 | 0.2 | 0.3 |
| 22:6 (n-3) | 11.3 | 0.4 | 0.5 | 0.4 | 0.6 |
| SAFA* | 30.5 | 10.4 | 10.6 | 10.0 | 10.1 |
| MUFA** | 22.3 | 21.7 | 21.6 | 17.6 | 17.6 |
| PUFA*** | 29.4 | 65.5 | 65.0 | 69.0 | 68.1 |
| (n-3) | 24.4 | 32.4 | 33.1 | 54.0 | 53.4 |
| (n-6) | 5.0 | 33.2 | 31.9 | 15.0 | 14.7 |
| (n-3)/(n-6) | 4.88 | 0.97 | 1.04 | 3.61 | 3.62 |

*saturated fatty acids include, among others, 14:0, 16:0, 18:0, 20:0, 22:0
**monounsaturated fatty acids include, among others, 16:1, 18:1 (n-9), 18:1 (n-7), 20:1, 22:1,
***polyunsaturated fatty acids include, among others, 18:2 (n-6), 18:3 (n-3), 20:5 (n-3), 22:6 (n-3)

Ten fishes were kept in each tank at a water temperature of 10° C. The fishes were fed ad libitum until their weight was doubled (30 days). At sacrifice, the muscle was divided in red and white muscle and muscles and liver were frozen at −80° C. until analyzed.

### Biochemical Analyses

Tissues and diets were extracted following the method of document [6]. Total lipids of tissues were separated into phospholipids (PL) and triacylglycerols (TG) according to the method disclosed in document [7]. Total lipids in the diets and the PL and TG lipid fractions of tissues were methylated following the procedure of document [8] and the fatty acids were analyzed with gas chromatograph CP3800 (Varian AB, Stockholm, Sweden) equipped with flame ionisation detector (FID) and split injector and fitted with a fused silica capillary column BPX 70 (SGE, Austin, Tex., length 50 m, inner diameter 0.22 mm, 0.25 μm film thickness). The column temperature was programmed to start at 158° C., hold 5 min and then increase 2° C./min from 158° C. to 220° C. and remain at 220° C. for 8 min. The carrier gas was helium (0.8 ml/min) and make up gas was nitrogen. The injector and detector temperatures were 230° C. and 250° C. respectively. Fatty acids were identified by comparison with the standard fatty acid mixture GLC-68. Peak areas were integrated using a Star chromatography workstation software version 5.5 (Varian AB, Stockholm, Sweden).

### Results

The growth of the fishes did not differ between the different experimental feeds. Furthermore, the total fat content of the muscle tissues and liver did not significantly change for the different feed treatments as is illustrated in Table III.

TABLE III
percentage of lipid content in muscle and liver for different feeds

| Tissue | FO | MO | MO + S | LO | LO + S |
|---|---|---|---|---|---|
| Red muscle | 17.0 ± 4.7 | 10.5 ± 2.2 | 13.3 ± 1.7 | 13.7 ± 4.6 | 13.7 ± 2.5 |
| White muscle | 1.7 ± 0.5 | 2.0 ± 0.5 | 2.0 ± 0.3 | 2.8 ± 0.7 | 2.1 ± 0.5 |
| Liver | 1.8 ± 0.3 | 4.2 ± 0.3 | 4.4 ± 0.9 | 3.8 ± 0.4 | 4.6 ± 0.6 |

The fat content was about 2% in the white muscle and about 15% in the red muscle. The phospholipid fraction (PL) represented about 25% of the white muscle lipids but only a few percent of the red muscle lipids. White muscle represents the main muscle part, approximately 95%, and the red muscle consists of the remaining 5%. Therefore, the triacylglycerol storage fat fraction (TG) is the important fraction in terms of the fatty acid composition of fish muscle as human food.

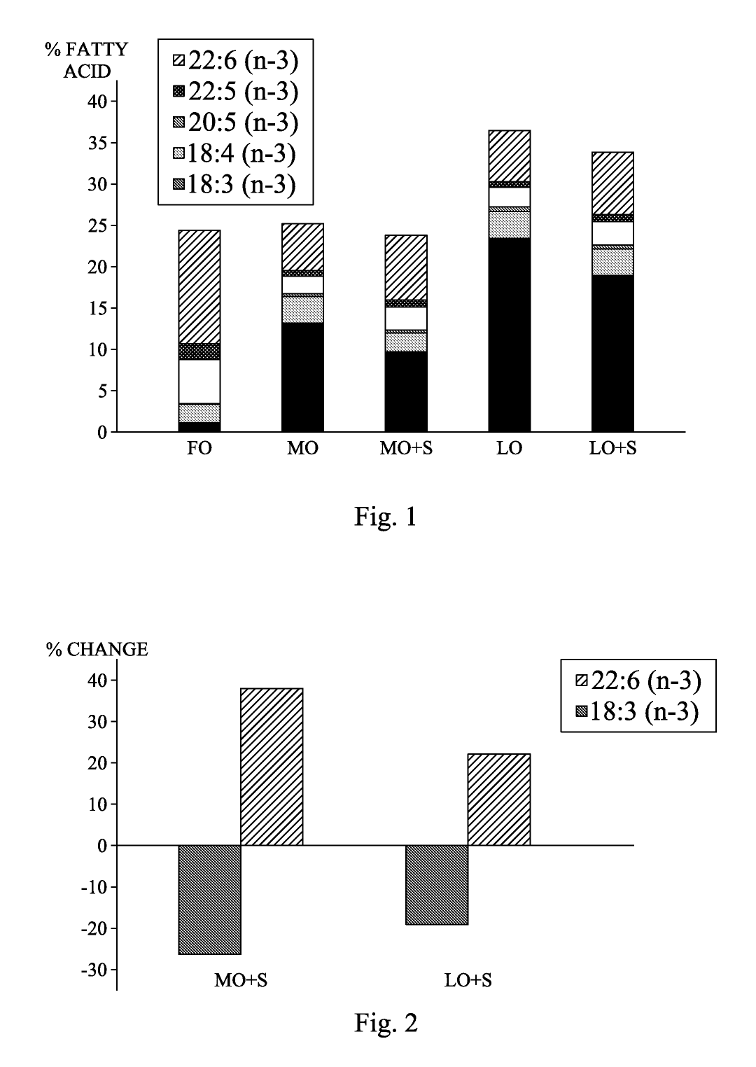

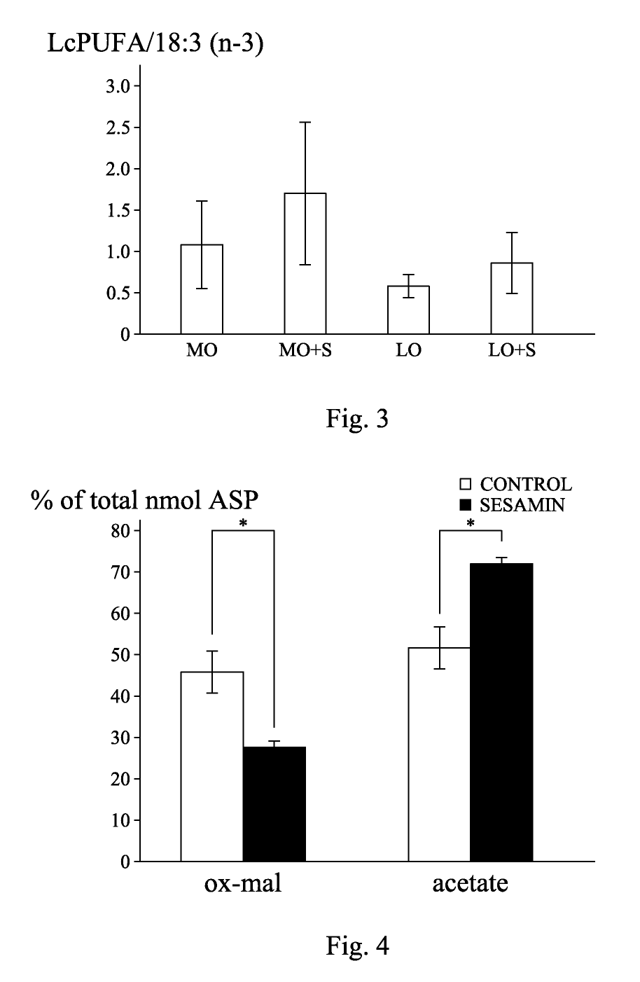

Some of the muscle fatty acids (18:3 n-3, 18:4 n-3, 20:4 n-3 and 22:6 n-3) have been significantly affected by the presence of sesamin/episesamin in the feed, especially the TG of the white muscle, see Table IV and FIGS. 1 and 2. The sesamin/episesamin diet increased the desaturation index for (n-3) fatty acids. Furthermore, the ratio of n-3 HUFA to 18:3 (n-3), i.e. the proportion of fatty acids with more than or equal to 20 carbons and at least 3 double bounds to α-linolenic acid, increased in the white muscle TG, see FIG. 3. In Table IV and V the following abbreviations have been used: SAFA=saturated fatty acids; MUFA=monounsaturated fatty acids; PUFA=polyunsaturated fatty acids; and HUFA=highly unsaturated fatty acids (≧3 double bounds)

However, as is illustrated in Table V, the liver fatty acid composition was not significantly altered in any of the two lipid fractions, TG and PL.

Triacylglycerols (TAG), the main fraction of fish muscle lipids, was most influenced by the sesamin/episesamin feeding. Sesamin/episesamin especially increased the proportion of DHA and decreased that of ALA, see FIG. 2. Thus, addition of the sesame lignans in the vegetable oil-containing feed increases the desaturation index in the oil fed fish as is illustrated in FIG. 3. The total amounts of (n-3) fatty acids and (n-6) fatty acids and the (n-3)/(n-6) fatty acid ratio were not significantly affected. Thus, the presence of sesamin/episesamin in fish diet affected the elongation and desaturation of 18:3 n-3 towards longer chain omega-3 fatty acids and in particular DHA (p<0.05) and to some extent that of 18:2 n-6 towards AA.

TABLE IV
white muscle fatty acid composition

| Lipid class | Triglycerides | | | | |
|---|---|---|---|---|---|
| Feed | FO | MO | MO + S | LO | LO + S |
| 18:2 (n-6) | 4.7 ± 0.2 | 21.8 ± 6.2 | 16.0 ± 4.1 | 10.4 ± 0.7 | 9.8 ± 1.2 |
| 18:3 (n-3) | 1.1 ± 0.1 | 13.2 ± 4.2 | 9.7 ± 3.5 | 23.4 ± 3.6 | 19.0 ± 4.4 |
| 18:4 (n-3) | 2.2 ± 0.3 | 3.2 ± 0.8 | 2.3 ± 0.2 | 3.3 ± 0.2 | 3.2 ± 0.4 |
| 20:2 (n-6) | 0.3 ± 0.0 | 0.5 ± 0.1 | 0.4 ± 0.1 | 0.3 ± 0.0 | 0.3 ± 0.0 |
| 20:3 (n-6) | 0.2 ± 0.0 | 0.4 ± 0.1 | 0.3 ± 0.1 | 0.2 ± 0.0 | 0.2 ± 0.0 |
| 20:4 (n-6) | 0.5 ± 0.0 | 0.3 ± 0.1 | 0.3 ± 0.0 | 0.2 ± 0.0 | 0.2 ± 0.0 |
| 20:3 (n-3) | 0.1 ± 0.1 | 0.3 ± 0.1 | 0.3 ± 0.1 | 0.6 ± 0.1 | 0.4 ± 0.1 |
| 20:4 (n-3) | 1.4 ± 0.1 | 2.8 ± 1.9 | 4.3 ± 1.2 | 3.1 ± 0.4 | 4.0 ± 0.8 |
| 20:5 (n-3) | 5.3 ± 0.1 | 2.2 ± 0.9 | 2.8 ± 0.5 | 2.4 ± 0.4 | 2.8 ± 0.4 |
| 22:5 (n-3) | 1.9 ± 0.1 | 0.7 ± 0.3 | 0.9 ± 0.3 | 0.7 ± 0.1 | 0.9 ± 0.1 |
| 22:6 (n-3) | 13.7 ± 0.9 | 5.7 ± 2.4 | 7.8 ± 1.6 | 6.2 ± 0.7 | 7.5 ± 1.0 |
| SAFA | 29.8 ± 1.4 | 15.8 ± 2.0 | 19.2 ± 1.9 | 17.2 ± 1.3 | 18.0 ± 1.8 |
| MUFA | 33.1 ± 0.5 | 28.4 ± 2.8 | 29.9 ± 2.0 | 26.3 ± 1.2 | 27.4 ± 1.8 |
| PUFA | 31.5 ± 1.3 | 50.9 ± 5.8 | 45.1 ± 4.6 | 50.7 ± 2.7 | 48.4 ± 3.8 |
| (n-3) | 25.8 ± 1.3 | 28.0 ± 0.9 | 28.09 ± 1.2 | 39.60 ± 2.1 | 37.87 ± 2.7 |
| (n-6) | 5.7 ± 0.1 | 22.9 ± 6.2 | 17.0 ± 4.2 | 11.1 ± 0.7 | 10.5 ± 1.2 |
| (n-3)/(n-6) | 4.3 ± 0.3 | 1.3 ± 0.4 | 1.7 ± 0.4 | 3.58 ± 0.0 | 3.62 ± 0.2 |
| (n-3) HUFA/ | 22.0 ± 1.3 | 1.3 ± 0.7 | 2.2 ± 1.1 | 0.7 ± 0.2 | 1.1 ± 0.5 |
| 18:3 (n-3) | | | | | |
| (n-6) HUFA/ | 0.22 ± 0.0 | 0.06 ± 0.0 | 0.06 ± 0.01 | 0.06 ± 0.01 | 0.07 ± 0.02 |
| 18:2 (n-6) | | | | | |

| Lipid class | Phospholipids | | | | |
|---|---|---|---|---|---|
| Feed | FO | MO | MO + S | LO | LO + S |
| 18:2 (n-6) | 1.3 ± 0.0 | 6.2 ± 2.0 | 4.8 ± 1.0 | 3.2 ± 0.2 | 2.8 ± 0.2 |
| 18:3 (n-3) | 0.4 ± 0.0 | 5.1 ± 1.2 | 5.0 ± 1.0 | 9.9 ± 1.0 | 8.4 ± 0.9 |
| 18:4 (n-3) | 0.4 ± 0.0 | 1.0 ± 0.3 | 0.7 ± 0.1 | 1.0 ± 0.1 | 0.9 ± 0.1 |
| 20:2 (n-6) | 0.1 ± 0.0 | 0.5 ± 0.2 | 0.4 ± 0.1 | 0.3 ± 0.0 | 0.2 ± 0.1 |
| 20:3 (n-6) | 0.2 ± 0.0 | 0.6 ± 0.3 | 0.5 ± 0.1 | 0.3 ± 0.0 | 0.2 ± 0.0 |
| 20:4 (n-6) | 1.1 ± 0.0 | 1.0 ± 0.3 | 0.8 ± 0.1 | 0.6 ± 0.0 | 0.7 ± 0.1 |
| 20:3 (n-3) | 0.0 ± 0.0 | 0.4 ± 0.1 | 0.4 ± 0.2 | 0.8 ± 0.1 | 0.6 ± 0.1 |
| 20:4 (n-3) | 0.9 ± 0.0 | 1.3 ± 0.3 | 1.4 ± 0.2 | 1.8 ± 0.1 | 1.7 ± 0.2 |
| 20:5 (n-3) | 7.1 ± 0.2 | 5.5 ± 0.5 | 5.1 ± 0.4 | 5.2 ± 0.2 | 5.6 ± 0.4 |
| 22:5 (n-3) | 1.7 ± 0.04 | 1.2 ± 0.1 | 1.2 ± 0.1 | 1.2 ± 0.1 | 1.3 ± 0.1 |
| 22:6 (n-3) | 48.9 ± 1.0 | 40.9 ± 3.3 | 43.9 ± 0.7 | 38.6 ± 2.6 | 43.7 ± 1.7 |
| SAFA | 29.0 ± 1.2 | 26.3 ± 1.6 | 27.1 ± 3.8 | 27.1 ± 2.7 | 24.9 ± 0.7 |
| MUFA | 8.7 ± 0.1 | 8.5 ± 0.8 | 7.6 ± 0.6 | 8.1 ± 0.3 | 7.3 ± 0.6 |
| PUFA | 62.2 ± 1.4 | 63.7 ± 1.5 | 64.0 ± 2.9 | 62.7 ± 3.0 | 66.2 ± 1.1 |
| (n-3) | 59.3 ± 1.3 | 55.4 ± 1.4 | 57.5 ± 1.7 | 58.4 ± 2.8 | 62.2 ± 1.2 |
| (n-6) | 2.6 ± 0.1 | 8.3 ± 2.7 | 6.5 ± 1.2 | 4.3 ± 0.3 | 4.0 ± 0.3 |
| (n-3)/(n-6) | 22.6 ± 0.4 | 7.2 ± 2.3 | 9.0 ± 1.6 | 13.2 ± 0.7 | 15.4 ± 1.3 |
| (n-3) HUFA/ | 14.5 ± 1.4 | 10.4 ± 3.0 | 10.9 ± 2.2 | 5.0 ± 0.6 | 6.3 ± 0.8 |
| 18:3 (n-3) | | | | | |
| (n-6) HUFA/ | 1.0 ± 0.02 | 0.34 ± 0.03 | 0.3 ± 0.03 | 0.36 ± 0.03 | 0.40 ± 0.03 |
| 18:2 (n-6) | | | | | |

n = 6 in all groups, significant differences (P < 0.05) are marked in bold

TABLE V
liver fatty acid composition

| Lipid class | Triglycerides | | | | |
|---|---|---|---|---|---|
| Feed | FO | MO | MO + S | LO | LO + S |
| 18:2 (n-6) | 6.6 ± 0.63 | 24.6 ± 2.6 | 26.4 ± 1.9 | 13.1 ± 0.9 | 14.1 ± 1.5 |
| 18:3 (n-3) | 6.0 ± 02.37 | 10.1 ± 1.7 | 12.3 ± 1.2 | 25.2 ± 2.1 | 23.6 ± 2.3 |
| 18:4 (n-3) | 1.72 ± 0.21 | 2.9 ± 0.6 | 2.4 ± 0.5 | 4.2 ± 1.2 | 3.7 ± 0.9 |
| 20:2 (n-6) | 0.3 ± 0.04 | 2.2 ± 0.8 | 2.1 ± 0.4 | 1.1 ± 0.4 | 1.1 ± 0.2 |
| 20:3 (n-6) | 0.2 ± 0.04 | 1.6 ± 0.4 | 1.6 ± 0.2 | 0.4 ± 0.2 | 0.5 ± 0.2 |
| 20:4 (n-6) | 0.6 ± 0.06 | 0.6 ± 0.1 | 0.4 ± 0.1 | 0.1 ± 0.07 | 0.2 ± 0.05 |
| 20:3 (n-3) | 0.05 ± 0.03 | 1.0 ± 0.4 | 1.3 ± 0.4 | 2.7 ± 1.1 | 2.4 ± 0.4 |
| 20:5 (n-3) | 6.4 ± 1.03 | 1.8 ± 0.6 | 2.2 ± 0.6 | 2.5 ± 0.5 | 2.6 ± 0.6 |
| 22:5 (n-3) | 2.2 ± 0.41 | 0.4 ± 0.2 | 0.6 ± 0.2 | 0.7 ± 0.2 | 0.6 ± 0.1 |
| 22:6 (n-3) | 13.4 ± 2.04 | 2.4 ± 1.4 | 3.9 ± 1.3 | 4.4 ± 0.8 | 4.3 ± 0.9 |
| SAFA | 21.1 ± 1.69 | 12.1 ± 4.7 | 9.4 ± 0.7 | 9.4 ± 1.6 | 9.6 ± 1.3 |
| MUFA | 36.7 ± 1.05 | 24.6 ± 2.8 | 30.9 ± 1.1 | 28.1 ± 1.7 | 29.8 ± 2.1 |
| PUFA | 37.4 ± 1.29 | 47.5 ± 6.1 | 53.0 ± 1.1 | 54.5 ± 3.0 | 52.9 ± 2.4 |
| (n-3) | 29.8 ± 1.18 | 18.6 ± 3.2 | 22.6 ± 1.3 | 39.8 ± 2.5 | 37.1 ± 1.4 |
| (n-6) | 7.7 ± 0.61 | 28.9 ± 3.0 | 30.4 ± 1.9 | 14.7 ± 1.2 | 15.8 ± 1.4 |
| (n-3)/(n-6) | 3.9 ± 0.38 | 0.64 ± 0.05 | 0.75 ± 0.1 | 2.7 ± 0.3 | 2.4 ± 0.2 |
| (n-3) HUFA/ | 4.5 ± 1.9 | 0.7 ± 0.2 | 0.7 ± 0.2 | 0.5 ± 0.05 | 0.5 ± 0.1 |
| 18:3 (n-3) | | | | | |
| (n-6) HUFA/ | 0.2 ± 0.03 | 0.2 ± 0.04 | 0.2 ± 0.02 | 0.12 ± 0.03 | 0.12 ± 0.02 |
| 18:2 (n-6) | | | | | |

| Lipid class | Phospholipids | | | | |
|---|---|---|---|---|---|
| Feed | FO | MO | MO + S | LO | LO + S |
| 18:2 (n-6) | 2.7 ± 0.3 | 7.6 ± 0.7 | 8.3 ± 0.4 | 4.9 ± 0.3 | 4.6 ± 0.2 |
| 18:3 (n-3) | 0.5 ± 0.06 | 2.6 ± 0.6 | 3.5 ± 0.3 | 8.4 ± 0.9 | 7.1 ± 0.8 |
| 18:4 (n-3) | 0.2 ± 0.01 | 0.6 ± 0.2 | 0.5 ± 0.1 | 1.2 ± 0.2 | 0.9 ± 0.3 |
| 20:2 (n-6) | 0.3 ± 0.04 | 1.1 ± 0.3 | 1.2 ± 0.3 | 0.7 ± 0.2 | 0.6 ± 0.1 |
| 20:3 (n-6) | 0.2 ± 0.03 | 1.5 ± 0.6 | 1.1 ± 0.1 | 0.7 ± 0.2 | 0.7 ± 0.2 |
| 20:4 (n-6) | 3.1 ± 0.3 | 2.4 ± 0.3 | 2.1 ± 0.3 | 1.0 ± 0.2 | 1.2 ± 0.4 |
| 20:3 (n-3) | 0.1 ± 0.06 | 0.4 ± 0.1 | 0.4 ± 0.1 | 1.2 ± 0.3 | 1.0 ± 0.1 |
| 20:5 (n-3) | 6.1 ± 0.3 | 4.0 ± 0.4 | 4.5 ± 0.5 | 6.8 ± 0.7 | 5.2 ± 0.6 |
| 22:5 (n-3) | 42.1 ± 1.09 | 0.8 ± 0.3 | 0.7 ± 0.2 | 0.8 ± 0.2 | 0.8 ± 0.1 |
| 22:6 (n-3) | 26.4 ± 1.3 | 39.7 ± 2.7 | 41.5 ± 1.9 | 39.6 ± 1.4 | 39.4 ± 1.2 |
| SAFA | 12.3 ± 0.7 | 24.7 ± 2.9 | 22.0 ± 1.1 | 21.1 ± 1.3 | 23.6 ± 1.6 |
| MUFA | 54.0 ± 0.9 | 11.1 ± 0.3 | 10.2 ± 0.5 | 9.7 ± 0.3 | 10.8 ± 0.2 |
| PUFA | 50.2 ± 1.1 | 60.8 ± 2.4 | 63.9 ± 1.6 | 65.2 ± 1.4 | 61.4 ± 1.8 |
| (n-3) | 6.2 ± 0.4 | 48.1 ± 2.5 | 51.2 ± 1.7 | 58.0 ± 1.4 | 54.4 ± 1.8 |
| (n-6) | 8.1 ± 0.7 | 12.7 ± 0.7 | 12.7 ± 0.7 | 7.2 ± 0.2 | 7.0 ± 0.5 |
| (n-3)/(n-6) | 0.013 ± 0.0 | 3.8 ± 0.3 | 4.05 ± 0.3 | 8.1 ± 0.3 | 7.78 ± 0.6 |
| (n-3) HUFA/ | 106.0 ± 12.2 | 18.1 ± 3.4 | 13.9 ± 1.8 | 5.9 ± 0.7 | 6.8 ± 0.9 |
| 18:3 (n-3) | | | | | |
| (n-6) HUFA/ | 1.4 ± 0.2 | 0.7 ± 0.1 | 0.5 ± 0.1 | 0.5 ± 0.1 | 0.6 ± 0.1 |
| 18:2 (n-6) | | | | | |

n = 6 in all groups, significant differences (P < 0.05) are marked in bold

This indicates that sesamin and episesamin can be added to feeds containing plant oils containing eighteen carbons, unsaturated fatty acids to improve their elongation and desaturation in aquaculture species towards long chain polyunsaturated fatty acids (LCPUFA), especially DHA, which is beneficial in relation to nutrition of both aquaculture species and humans. This possibility of chain elongation and desaturation will increase the efficacy of alternate plant oil partial replacement to the oil from fresh and marine water species needed in aquaculture production.

The total amount of PUFA in sesamin fed aquaculture species is lower than in species fed diets without sesamin. Previously sesamin has been reported to increase β-oxidation, which may be the mechanism causing a lower PUFA content in sesamin fed species.

Sesame lignans were also tested for possible interactions in lipid metabolism in Atlantic salmon (Salmo salar L.) hepatocytes. Incubation with the lignans resulted in the increased percentage of DHA and clearly increased (β-oxidation, elongation and desaturation of 18:3 (n-3). The main β-oxidation product of the acid soluble fraction was acetate. The DHA levels elongated and desaturated from 18:3 (n-3) were twice as high when hepatocytes were incubated with the lignans compared to the control group. The amount of secreted triacylglycerol was lower in lignan-incubated hepatocytes. Incubation with the lignans also showed effects of expression on some lipid related genes.

### Chemicals

The radiolabeled FA [1-¹⁴C] 18:3 (n-3) (specific radioactivity 50 mCi/mmol) was obtained from American Radiolabelled Chemicals (St. Louis, Mo., USA). The non-labeled 18:3 (n-3), essential fatty acid free bovine serum albumin (BSA), fetal bovine serum (FSB), Leibowitz-15 (L-15), HEPES, 2′,7′-dichlorflourescein, 2′,7′-dichlorflourescin and collagenase type 1 dimethyl sulfoxide (DMSO), phosphate buffered saline (PBS), phenylethylamine and 4-(2-hydroxyethyl)-1-piperazineethanesulfonic acid (HEPES), ethylenediamine-tetraacetic acid (EDTA), sodium bicarbonate solution, L-glutamine, were obtained from Sigma Chemical Co. (St. Louis, Mo., USA) and methacain MS-222 from Norsk Medisinaldepot (Norway). Sesamin (Sesamin/episesamin mixture, 1:1, w/w) was a kind gift from Takemoto Oil and Fat Co., Ltd. (Gamagori Aichi, Japan). Fatty acid peaks were identified by comparison with the standard mixture GLC-68 A (Nu-check Prep, Inc, Elysian, Minn., USA). Ammonium dihydrogenphosphate, Perchloric acid (HClO₄), TLC-plates, all solvents and other chemicals for fatty acid and sesamin analysis and were purchase from Merck (Darmstadt, Germany).

### Isolation of Hepatocytes

Atlantic salmon of approximately 800 g from NIVAs Research Station, Drøbak, Norway, kept in seawater at 10° C. and fed a commercial diet, restricted feeding was used for isolation of hepatocytes. Hepatocytes were isolated from three fishes at three different time points. The liver was perfused following a two-step collagenase procedure developed by Seglen [11] and modified by Dannevig and Berg [12].

Incubation of Hepatocytes with Linolenic Acid 18:3 (n-3)

The isolated hepatocytes were incubated with 35 nmol, 2.5 μCi of [1-¹⁴C] 18:3 (n-3) as well as non radiolabeled 18:3 (n-3), final concentration 7 μM, in 5 ml of L 15 culture medium without FBS. The radio labeled FA was added to the medium as potassium salt bound to BSA. To each experimental flask (4 radiolabeled and 7 with non-radiolabeled 18:3 (n-3)), DMSO (7.4 μl/5 ml) with sesamin (final concentration 0.05 mM) was added and to each control flasks (4 radiolabeled and 7 with non-radiolabeled 18:3 (n-3)), only DMSO (7.4 μl/5 ml) was added, in another two flasks sesamin but no FA was added. The cells were incubated at 12° C. for 48 h. The radiolabeled experiment flasks with and without sesamin (4 each) were washed and harvested in PBS for further analysis of β-oxidation and radiolabeled lipid classes and radiolabeled cellular fatty acids. The cells in the two flasks with no FA substrate added were denaturated after the incubation and thereafter added radiolabeled substrate in order to be used as a negative control for the ASP measurements and HPLC analyses. In the non-labeled study, control and sesamin flask (4 each) were washed in PBS prior to isolation of mRNA according to the protocol RNeasy® Mini Kit for gene expression analysis and 3 flasks of each control and sesamin were washed and harvested in PSB for fatty acid and sesamin analysis.

### Incubation of Hepatocytes for Dose Response and Time Course Study

The cells for the dose response study were washed in PBS 24 h after seeding and incubated in L 15 culture medium with FBS (the fatty acid composition of L15+FBS was: 14:0=8.4; 14:1=0.6; 15:0=2.3; 16:0=31.7; 16:1=4.1; 18:0=31.7; 18:1 (n-9)=14.9; 18:1 (n-7)=2.7; 18:2 (n-6)=3.7; 18:3 (n-3)=1.0; 20:3 (n-6)=0.8; 20:4 (n-6)=2.4; 20:5 (n-3)=0.5; 22:5 (n-3)=0.9; 22:6 (n-3)=1.2) and with 5 different concentrations of sesamin. Sesamin solved in DMSO (7.4 μl/5 ml medium) was added the flasks with final concentrations of: 0.005 mM, 0.05 mM, 0.075 mM and 0.11 mM to duplicate flasks of cells in a regression design and incubated for 42 h at 12° C. For the time study the final concentration of 0.05 mM sesamin in culture medium were added to the hepatocytes in duplicates flasks. The cells were incubated for 0, 18, 24, 42, 48, 66 and 72 h. As control in both the dose response study and the time study DMSO (7.4 μl/5 ml medium) was added the cells. After incubation, the cells were washed in PBS prior to isolation of mRNA according to the protocol RNeasy® Mini Kit for gene expression analysis.

### Analysis of Lipids in Cells and Media

Lipids from cells and media incubated with [1-¹⁴C] 18:3 (n-3) were extracted according to Folch et al. [13] and the fatty acid composition of the cells was determined by RP-HPLC as described by Narce et al. [14]. The mobile phase was acetonitrile:H₂O (85:15 v/v) with a flow rate of 1 ml/min and a temperature of 30° C. The column used was a revere phase Symmetry 3.5 μm C₁₈ column and the FAs were detected with a radioactive detector A-100 (Radiomatic Instrument & Chemicals, Tampa, Fla., USA). The FAs were identified by comparison with external standards and retention times. The lipids extracted from the media were separated on TLC-plates into lipid classes using a mixture of petroleum ether, diethyl eter and acetic acid (113:20:2 v/v/v). The lipid classes, phospholipids (PL), monoglycerides (MG) free fatty acids (FFA) and triacylglycerols (TAG) were developed by spraying 0.2% 2′,7′-dichlorofluorescein in methanol and identified by compassion to standards under UV-light. The lipid classes were scraped off and dissolved in scintillation fluid for scintillation counting (TRI-CARB 1900 TR, Packard Instruments). The esterified fatty acids, PL, MG and TAG fraction will be denoted as secreted fatty acids.

Lipids from the none-radioactive cells and media used for the time course gene expression study were extracted by using hexane:isopropanol (3:2 v/v). Total lipids were converted to fatty acid methylesters by using BF3 and analysed with Gas Chromatograph CP3800 (Varian AB, Stockholm, Sweden) according to Fredriksson et al. [15].

Measurements of β-oxidation

The capacity of β-oxidation was measured by determination of the oxidation products ¹⁴C acid soluble products (ASP) and ¹⁴C CO₂ as described by Christiansen et al. [16]. ¹⁴C CO₂ produced during the incubation of the cells was measured by transferring 1.5 ml of the media to glass vials sealed with a rubber stop and a central well containing a Whatman filer paper with 0.3 ml phenylethylamine/methanol (1:1, v/v). The medium were acidified with 0.3 ml 1M HClO₄ and ¹⁴C CO₂ was trapped for 1 h. Then the filer papers were placed in vials and dissolved in 8 ml of scintillation fluid for scintillation counting (Radiomatic Instrument & Chemicals, Tampa, Fla., USA). The amount of ¹⁴C ASP produces were determined by adding 0.5 ml ice cooled 2M HClO₄ to the incubation media and incubated at 4° C. for 1 h, then the samples were centrifuged at 17 950×g for 10 min. at 10° C. and 200 μl of the supernatant was collected for scintillation counting (Radiomatic Instrument & Chemicals, Tampa, Fla., USA). The remaining supernatant was neutralised with NaOH and the different ASP were detected by using high pressure liquid chromatography (HPLC) equipped with a ChromSep Inertsil C8-3 column (250 mm×4.6 mm stainless steel), an UV-detector at 210 nm and radioactive detector A-100 (Radiomatic Instrument & Chemicals, Tampa, Fla., USA) coupled to the UV detector. The mobile phase was 0.1 M ammonium dihydrogenphosphate adjusted with phosphoric acid to pH 2.5, the flow rate was 1 ml/min. The components were identified by comparison to external standards and retention times.

### Protein Determination and ACO Activity

The protein content of the cells was determined by using the total protein kit (Micro Lowry/Peterson's modification) [17, 18] and measured at 540 nm in a 96 well plate reader Titertek, Multiscan (Labsystem, Finland). The ACO (acyl-CoA oxidase) activity was determined according to Small et al. [19]. The oxidation of 2,7 dichloroflourescin (LDCF) was measured with GBC UV/VIS 98 (GBC Scientific equipment, PTY, LTD, Melborne, Victoria, Australia) spectrophotometer (502 nm) during 3 min. The assay mixture contained 10 μl LDCF, 50 μl peroxidase type II in 0.1 M TRIS, 10 μl BSA (60 mg/ml), 10 μl FAD, 0.15 mg protein and H₂O up to 1 ml. The reaction was started with 10 μl PalmCoA (6 mg/ml 0.1 M TRIS).

Total RNA Extraction and cDNA Synthesis and Real Time PCR

Total RNA was isolated from the cells using the RNeasy® Mini Kit with on-column Rnase free Dnase set (Qiagen, MD, USA). All protocols were according to the manufacture's instructions. Purity and density were measured by optical density (NanoDrop® ND-1000 spectrophotometer, Saveen Werner, Limhamn, Sweden) and samples were stored in RNase-free water (Eppendorf, Hamburg, Germany) at −70° C.

The cDNA was synthesized following a modified protocol from the Tali Man Reverse Transcription Reagents kit (Applied Biosystems). The Oligo d(T)16 primers were used. The reaction was preformed by incubating the samples at 25° C. for 10 min, 48° C. for 6 min, 95° C. for 50 min and was terminated by reducing temperature to 10° C.

Primers for Real-Time PCR analysis (Table VI) were designed using the Primer Express® software and ordered from Invitrogene (CA, USA). They were designed on available salmon sequences in the GenBank®

TABLE VI
Sequence of primers

| | | | | | |
|---|---|---|---|---|---|
| Primer | Forward primer (5′-3′) | Reverse primer (5′-3′) | Acc. no. | Effic. | Seq id no |

| | | | | | |
|---|---|---|---|---|---|
| RPL2 | TAACGCCTGCCTCT- | ATGAGGGACCTTGTA- | CA049789 | 1.95 | 1, 2 |
| | TCACGTTGA | GCCAGCAA | | | |
| EF1A | CACCACCGGCCATC- | TCAGCAGCCTCCTTC- | AF321836 | 1.97 | 3, 4 |
| | TGATCTACAA | TCGAACTTC | | | |
| SsaPPARβ | CCAGCAACCCGTCC- | GAGACGGTCAGGGA- | AJ416953 | 2.04 | 5, 6 |
| | TTGTT | GCTCAC | | | |
| SsaPPARα | CGTTGAATTTCATGG- | TCCTGGTGGCCTAC- | DQ294237 | 1.90 | 7, 8 |
| | CGAACT | GGATC | | | |
| SalPPARγ | CATTGTCAGCCTGTC- | ATGTGACATTCCCA- | AJ292963 | 1.95 | 9, 10 |
| | CAGAC | CAAGCA | | | |
| ssalSRB | AACTCAGTGAAGAG- | TGCGGCGGTGATG- | DQ266043 | 1.79 | 11, 12 |
| | GCCAAACTTG | ATG | | | |
| cd36 | GGATGAACTCCCTG- | TGAGGCCAAAGTACT- | AY606034 | 1.76 | 13, 14 |
| | CATGTGA | CGTCGA | | | |
| Δ-5 | GAGAGCTGGCACCG- | GAGCTGCATTTTTCC- | AF478472 | 1.77 | 15, 16 |
| | ACAGAG | CATGG | | | |
| Δ-6 | AGAGCGTAGCTGAC- | TCCTCGGTTCTCTCT- | AY458652 | 1.90 | 17, 18 |
| | ACAGCG | GCTCC | | | |
| aco f1 | CCTTCATTGTACCTC- | CATTTCAACCTCATC- | DQ364432 | 2.00 | 19, 20 |
| | TCCGCA | AAAGCCAA | | | |
| Cpt1 f1 | GTACCAGCCCCGAT- | TCTCTGTGCGACCC- | AM230810 | 1.90 | 21, 22 |
| | GCCTTCAT | TCTCGGAA | | | |

Real-Time PCR for the dose response study was preformed in a Prism® 7000 system and gene-specific primers for SRB (sterol regulatory element binding protein), PPARα,β,γ, (peroxisome proliferator-activated receptor alpha, beta, gamma) and cd36 (Table VI). A 2×SYBR® Green PCR Mastermix (ABI). A reaction volume of 25 μl and 1 μl primers and 5 μl cDNA were used. All samples were analyzed in duplicate with non-template control on each plate. EF1A and RPL were used as reference genes. The reaction was preformed by incubating the samples at 50° C. for 2 min, 95° C. 10 min and 50 cycles of 95° C. for 10 s and 60° C. for 15 s. Standard curves were made for each primer pair and efficiency (E) were calculated E=10^{(-1/slope)}.

The Real-Time PCR for the time study and the cells incubated with non radio labeled 18:3 (n-3) was preformed in a LightCycler® 480 system using SYBR® Green PCR Mastermix (Roche, Oslo, Norway) and gene-specific primers for SRB, Δ-5, Δ-6, PPARα, β, γ, cd36, aco and cpt1 (carnitine palmiotyltransferase I) (Table VI). The PCR reaction mix (10 μl) consisted of 1 μl forward and reverse primer, 4 μl cDNA and 5 μl mastermix. All samples were analyzed in duplicate with non-template control on each plate and for each gene. RPL was used as a reference gene. The reaction was incubated at 95° C. for 5 min, 45 cycles of 95° C. for 10 s, 60° C. for 15 s and 72° C. for 15 s. Standard curves were made and the primer efficiency (E) was calculated for each primer pair using the LightCycler® 480 software.

### Statistical Analysis

Relative expressions of the different genes, in relation to housekeeping genes were determined using the Relative Expression Software Tool (REST-384©-version 1). For all other data, the student t-test in Microsoft Office Excel 2003 was used.

### Results

Lipid Content and Fatty Acid Profiles in Hepatocytes Incubated with 18:3 (n-3)

The total lipid content of the cells was 1.7 mg in the control cells and 1.4 mg in the sesamin cells (1.3% and 1.0% respectively), with no significant difference between the groups. Table VII shows the total fatty acid profiles of the hepatocytes incubated with non-radiolabeled 18:3 (n-3) in the absence and presence of sesamin (n=3 in control and sesamin group). The addition of sesamin significantly reduced the saturated fatty acids (SAFA, p<0.01) and the monounsaturated fatty acids (MUFA, p<0.02) and elevated the n-3 polyunsaturated fatty acids (p<0.01). Especially, the proportion of DHA was significantly higher in the sesamin-incubated hepatocytes, from 27.2% to 36.0% (p<0.01) while the proportion of EPA did not change. The desaturation index (n-3 LCPUFA/18:3n-3) was increased from 27.0 to 33.8 in the presence of sesamin (p<0.05).

TABLE VII
composition in total lipid fraction in hepatocytes

| | Control | Sesamin | *P-value |
|---|---|---|---|
| 14:0 | 1.77 ± 0.41 | 1.26 ± 0.02 | 0.10 |
| 15:0 | 0.46 ± 0.17 | 0.28 ± 0.01 | 0.14 |
| 16:0 | 21.4 ± 1.73 | 16.6 ± 0.84 | 0.01 |
| 16:1 | 1.30 ± 0.31 | 1.19 ± 0.05 | 0.58 |
| 18:0 | 4.84 ± 0.50 | 4.32 ± 0.28 | 0.20 |
| 18:1 (n-9) | 14.9 ± 0.56 | 12.6 ± 0.60 | 0.01 |
| 18:1 (n-7) | 2.13 ± 0.08 | 1.86 ± 0.09 | 0.02 |
| 18:2 (n-6) | 4.23 ± 0.28 | 3.87 ± 0.23 | 0.16 |
| 18:3 (n-3) | 1.66 ± 0.09 | 1.55 ± 0.12 | 0.28 |
| 20:0 | 0.26 ± 0.04 | 0.25 ± 0.04 | 0.73 |
| 20:1 | 1.11 ± 0.06 | 1.04 ± 0.05 | 0.15 |
| 20:2 (n-6) | 0.49 ± 0.04 | 0.47 ± 0.03 | 0.54 |
| 20:3 (n-6) | 0.48 ± 0.06 | 0.47 ± 0.04 | 0.74 |
| 20:4 (n-6) | 2.32 ± 0.87 | 2.77 ± 0.24 | 0.43 |
| 22:1 | 1.02 ± 0.11 | 1.07 ± 0.14 | 0.68 |
| 20:5 (n-3) | 7.90 ± 0.50 | 7.73 ± 0.49 | 0.69 |
| 22:5 (n-3) | 2.64 ± 0.33 | 2.76 ± 0.19 | 0.60 |
| 22:6 (n-3) | 27.2 ± 2.43 | 36.0 ± 2.76 | 0.01 |
| (n-3) | 39.4 ± 2.90 | 48.1 ± 1.98 | 0.01 |
| (n-6) | 7.52 ± 0.82 | 7.58 ± 0.49 | 0.92 |
| SAFA | 28.7 ± 1.73 | 22.7 ± 1.12 | 0.01 |
| MUFA | 20.5 ± 0.87 | 17.8 ± 0.87 | 0.02 |
| PUFA | 46.9 ± 3.12 | 55.7 ± 1.54 | 0.01 |
| (n-3) LCPUFA/18:3 (n-3) | 27.0 ± 2.77 | 33.8 ± 2.79 | 0.04 |

SAFA = saturated fatty acids
MUFA = monounsaturated fatty acids
PUFA = polyunsaturated fatty acids
(n-3) LCPUFA = 20:5 (n-3) + 22:5 (n-3) + 22:6 (n-3)
*P-value present comparison of sesamin and control group.

The proportions of radioactive fatty acids in hepatocytes are shown in Table VII (n=4 in each group). The percentage of DHA was significantly higher in the sesamin treated group than in the control group, 20.2% versus 10.5% (p<0.001). The percentages of 18:4 (n-3) (p=0.04) and the total amount of LCPUFA (p=0.01) were also higher in the sesamin group. Notably, the percentage of EPA was higher in the control than in the sesamin group, 12.3% versus 7.5% respectively (p<0.001). As with the none-labeled 18:3 (n-3) experiment, the desaturation index (LCPUFA/18:3 (n-3)) was significantly higher in the sesamin group (p=0.03).

TABLE VIII
proportion of radioactive FA in hepatocytes

| | Control | Sesamin | P-value |
|---|---|---|---|
| FA | Cells | | |
| 18:4 (n-3) | 1.99 ± 0.35 | 2.68 ± 0.38 | 0.04 |
| 20:5 (n-3) | 12.3 ± 0.85 | 7.53 ± 0.93 | <0.01 |
| 18:3 (n-3) | 57.7 ± 0.27 | 52.8 ± 4.57 | 0.07 |
| 22:6 (n-3) | 10.5 ± 0.10 | 20.2 ± 2.05 | <0.01 |
| 20:4 (n-3) | 2.72 ± 0.20 | 2.54 ± 0.28 | 0.32 |
| 20:3 (n-3) | 11.6 ± 0.37 | 10.6 ± 1.21 | 0.15 |
| 24:5 (n-3) | 0.61 ± 0.25 | 1.39 ± 0.28 | 0.01 |
| 16:0 | 2.54 ± 0.33 | 2.35 ± 0.52 | 0.54 |
| (n-3) LCPUFA/18:3 (n-3) | 0.49 ± 0.01 | 0.66 ± 0.12 | 0.03 |

(n-3) LCPUFA = 18:4 (n-3) + 20:4 (n-3) + 20:5 (n-3) + 24:5 (n-3) + 22:6 (n-3)

Distribution of ¹⁴C18:3 (n-3) Substrate in Cellular and Secreted Lipids, Lipid Classes and β-Oxidation Products

The total amount of ¹⁴C18:3 (n-3) in cells and media and the disruption of radiolabeled fatty acids in cellular, secreted lipids (i.e. all esterified lipid classes including PL, MG, TAG) and free fatty acids, as well as the amount of radiolabeled fatty acids in the β-oxidation products (ASP, CO₂ and total β-oxidation products=ASP+CO₂) are shown in Table IX (n=4 in each group). Significantly more ¹⁴C18:3 (n-3) have been taken up into the sesamin-incubated hepatocytes than into the control hepatocytes, 3.2 and 2.8 nmol fatty acids mg protein-1, respectively (p<0.001). Also the amount of secreted lipids (PL+TAG+MG) was lower in sesamin treated cells than the control cells (0.10±0.02 and 0.20±0.07 respectively, P=0.09), due to lower TAG content in the medium (Table IX). The addition of sesamin increased the total β-oxidation (ASP+CO₂), (p=0.006) of 18:3 (n-3) and especially the amount of ASP increased from 0.032±0.017 to 0.08±0.02 (p=0.006). The compound increased by sesamin in the ASP fraction was the acetate being 53.5% of ASP in the control group and 72.6% of ASP in the sesamin group (P<0.001), correspondingly the oxaloacetate and malate decreased, see FIG. 4. No significant differences in the enzymatic activity of acetyl CoA-oxidase between the groups were found, being 0.35±0.08 and 0.39±0.02 m unit mg protein-1 in control respectively sesamin cells.

TABLE IX
total 18:3 (n-3) in cells and media (in nmol), and distribution of FA
in cells and different medium fractions (nmol mg protein⁻¹)

| | Control | Sesamin | P-value |
|---|---|---|---|
| Total FA in cell and media | 23.63 ± 0.53 | 24.16 ± 0.28 | 0.13 |
| Cellular lipids | 2.80 ± 0.10 | 3.19 ± 0.03 | <0.01 |
| CO₂ in media | 0.007 ± 0.001 | 0.006 ± 0.000 | 0.06 |
| ASP in media | 0.032 ± 0.017 | 0.080 ± 0.020 | <0.01 |
| Total β-oxidation | 0.039 ± 0.017 | 0.087 ± 0.016 | <0.01 |
| FFA in media | 0.11 ± 0.05 | 0.11 ± 0.04 | 0.94 |
| *Secreted lipids in media | 0.20 ± 0.07 | 0.10 ± 0.02 | 0.09 |
| Total FA in cell and media | 23.63 ± 0.53 | 24.16 ± 0.28 | 0.13 |

*Secreted = triacylglycerols, monoglycerides and phospholipids in the medium

### Gene Expression

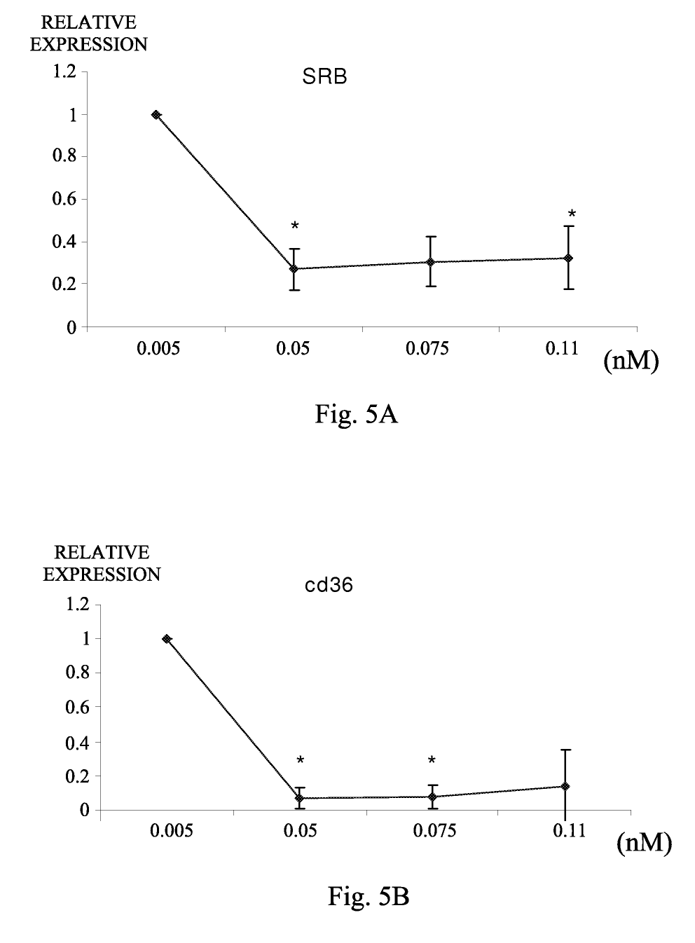

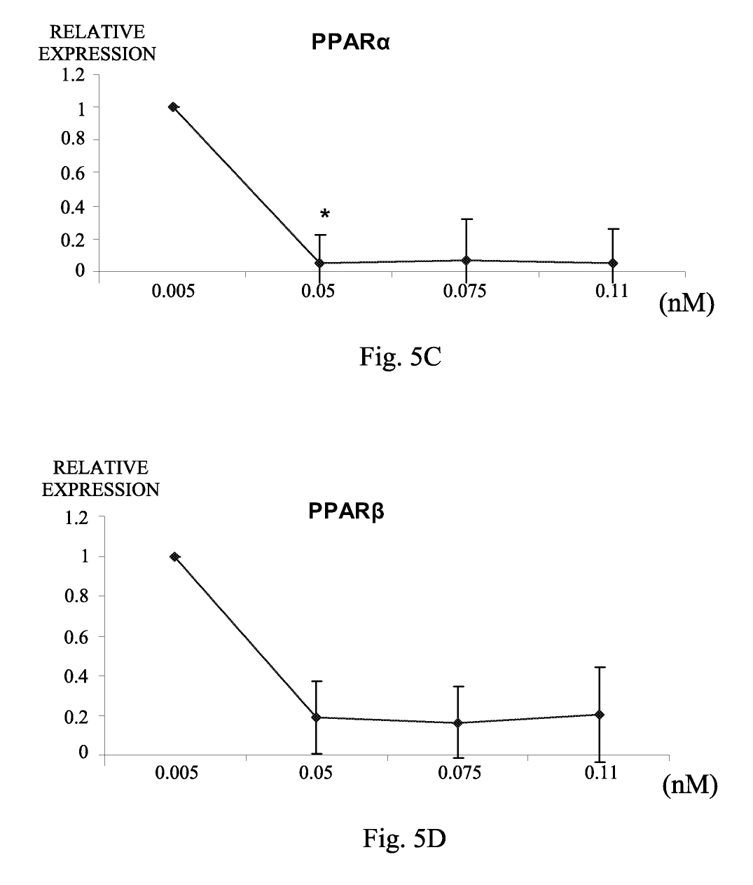

In the dose response experiment the relative expression of SRB, PPARα, PPARγ and CD36 were significantly down regulated at a concentration of 0.05 mM, when the concentration was increased to 0.075 mM only CD36 was significantly down regulated and at a dose of 0.11 mM SRB and PPARγ were significantly down-regulated, see FIGS. 5A to 5E. The lowest concentration 0.005 mM had no effect.

In the cells incubated with 18:3 (n-3) the expression of ctp1 was significantly upregulated by a factor 3.8, (p=0.001) and expression of PPARγ was downregulated by a factor 2.2 (p=0.001). No other genes showed significant changes.

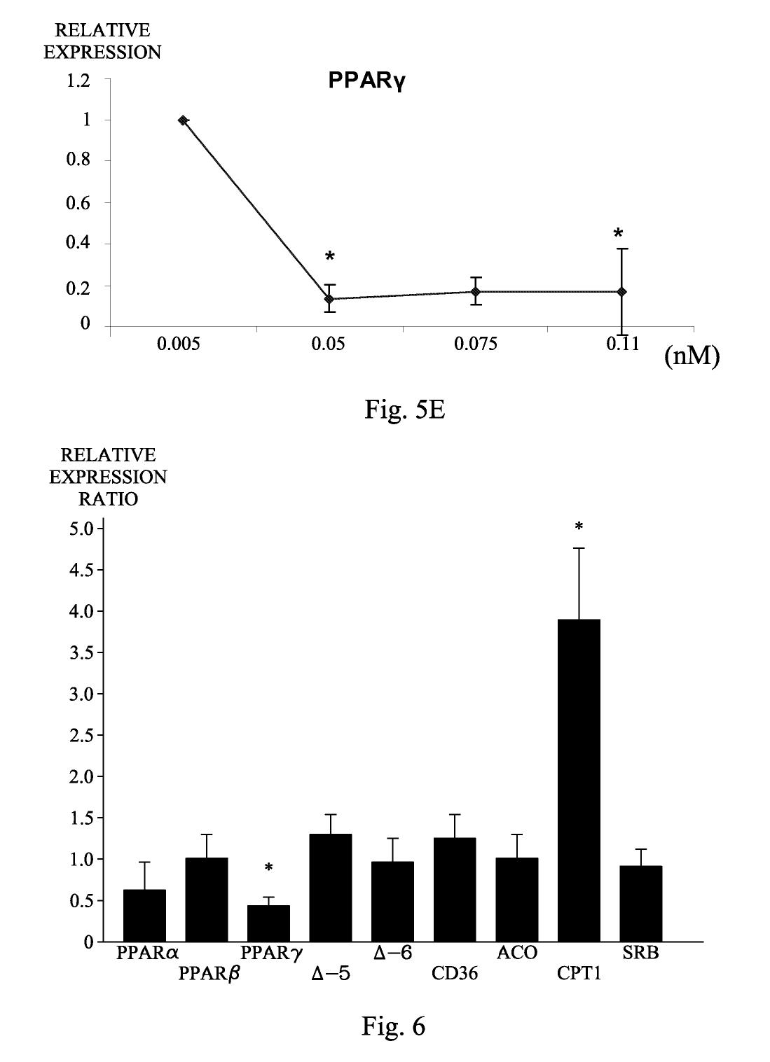

In the time study, significant effects of sesamin on several of the analyzed genes were found at different time points. The relative expression for Δ-5 and Δ-6 desaturases was significantly lower at several time points. FIG. 6 shows relative expression of respectively gene for each group. The relative expression decrease in the sesamin treated cells during the first 42 h. Both Δ-5 and Δ-6 desaturases followed the same pattern of relative expression.

FIG. 6 illustrates relative expression of some lipid related genes in salmon.

In the cells incubated with non-radioactive 18:3 (n-3) and sesamin the percentage of DHA (P<0.01), PUFA (P=0.01) and desaturation index (LCPUFA/18:3 (n-3)) (P=0.04) were significantly higher, and the percentage of 16:0 was significantly lower (P<0.01). In both the sesamin and the control cells incubated with ¹⁴C18:3 (n-3) elongation and desaturation occurred. However the elongation and desaturaturation to DHA was 100% more effective in and the desaturation index (LCPUFA/18:3 (n-3)) was significantly higher in the sesamin incubated cells (Table VIII). In the sesamin cells the amount of EPA is less, indicating that sesamin has a higher effect on Δ-6 desaturase than Δ-5 desaturase. These results support the previous in vivo finding disclosed above where significant increase of DHA was found in the white muscle of rainbow trout after feeding sesamin.

In this study we could demonstrate that sesamin addition increases the (β-oxidation of 18:3 (n-3) in salmon hepatocytes. Sesamin addition to salmon hepatocytes increased the proportion of acetate in the acid soluble product (ASP) fraction. The ACO-activity, another measurement of peroximal (β-oxidation showed down regulation in the sesamin group at 42 and 72 h. However, the analysis of enzymatic activity of acyl-CoA did not show any significant differences between the groups, but a slight increase in the sesamin group. The peroximal β-oxidation is also involved in the synthesis of DHA (shortening of 24:6 (n-3) to 22:6 (n-3)). Therefore, the increased DHA levels on this study could also indicate increased peroximal β-oxidation. On the other hand the increased expression of cpt1 indicates mitochondria (β-oxidation. As only the radioactive labeled products are found in the (β-oxidation analysis one can suggest that 18:3 (n-3) and its elongated and desaturated products were manly oxidized in the peroxisomes and that (β-oxidation of other fatty acids can be increased in the mitochondria. In the sesamin treated cells incubated with non radiolabeled 18:3 (n-3), lower percentage of 16:0 was found, possibly due to increased mitochondrial β-oxidation.

Sesamin decreased the amount of secreted lipids in the media with 53% from 0.20 to 0.10 nmol mg protein-1 (p=0.09), the most decreased lipid class was TAG. This indicates lower levels of very low-density lipoprotein (VLDL) secreted from the liver. In this study we showed a downregulation of PPARγ, which can indicate a reduced lipid synthesis. The expression of CD36, a gene involved in lipid storage and uptake is regulated by PPARγ and was also downregulated in the dose study at 0.05 and 0.075 mM sesamin. This may also affect the content of TAG in secreted lipids.

The sesamin content in the cells was only a small proportion of the given amount of sesamin to the cells (data not shown). The bioavailability of episesamin was higher than that of sesamin.

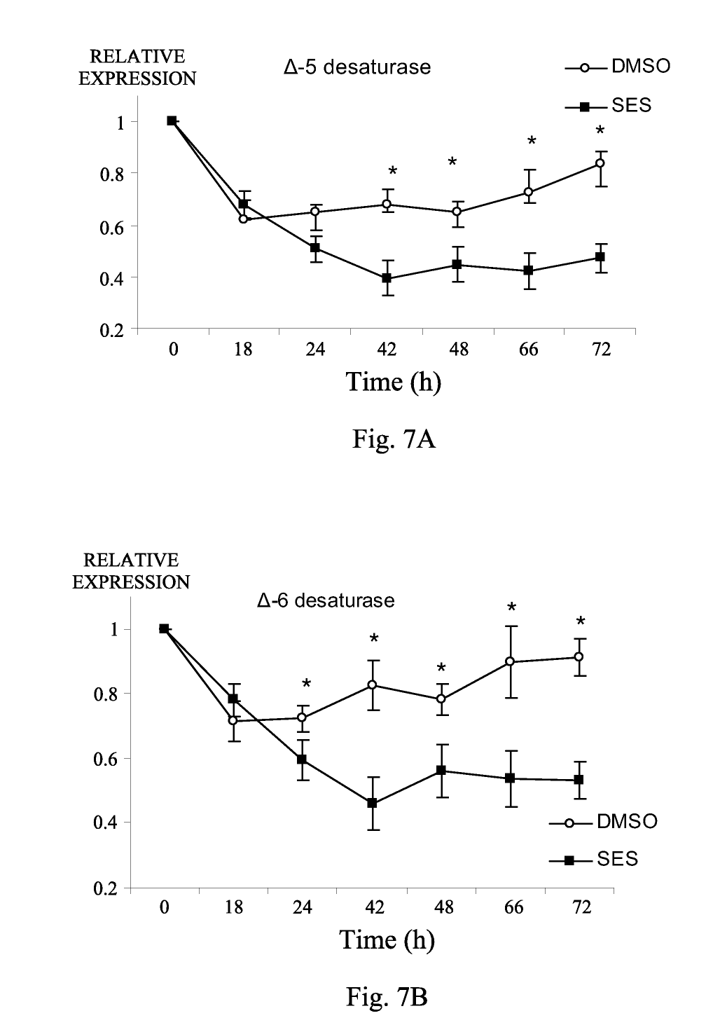

The relative expression of both Δ-5 and Δ-6 desaturase was significantly downregulated by sesamin at several time points. At all time points from 42 h to 72 h the relative expression of Δ-5 and Δ-6 desaturase was lower in the sesamin cells compared to the control cells (FIGS. 7A and 7B). One could speculate that the lower levels of mRNA are due to increased translation at a higher rate than the transcription rate. Interestingly the curve for both Δ-5 and Δ-6 desaturase for the sesamin cells follow the same pattern over time and is different compared to the control cells. However, the results clearly show that there is a clear effect on the expression of Δ-5 and Δ-6 desaturase and that sesamin significantly increase the desaturation and elongation from 18:3 (n-3) to DHA.

The above present experimental results provide strong evidence for that sesamin is a potent modulator of fatty acid metabolism in salmon hepatocytes. It significantly increased desaturation and elongation of 18:3 (n-3) towards DHA, resulting in twice as much DHA in the sesamin group as in the control group. The β-oxidation of lipids was clearly increased as well as the expression of cpt1, a gene involved in β-oxidation. Furthermore, sesamin has effect on several lipid related genes such as Δ-5 and Δ-6 desaturase and it decreases secretion of lipid from hepatocytes, in particular TAG-levels.

It will be understood by a person skilled in the art that various modifications and changes may be made to the present invention without departure from the scope thereof, which is defined by the appended claims.

### REFERENCES

[1] Bakthavathsalam et al., “Effect of different supplementary feeds on the growth rate of Indian major carp Catla catla reared under field conditions”, Environment & Ecology, 20: 833-838, 2002
[2] Bakthavathsalam et al., “Effect of different feeds on the growth rate of Indian major carp Catla catla kept under laboratory conditions”, Environment & Ecology, 20: 852-857, 2002
[3] Mazid et al., “Formulation of cost-effective feeds from locally available ingredients for carp polyculture system for increased production”, Aquaculture, 151: 71-78, 1997
[4] Moazzami, “Sesame seed lignans—diversity, human metabolism and bioactivites”, Doctorial Thesis No. 2006:98, Faculty of Natural Resources and Agricultural Sciences, Department of Food Science, Uppsala, Sweden
[5] Sanchez-Vaszquez et al., “Macronutrient self-selection through demand-feeders in rainbow trout”, Physiology & Behavior, 66: 45-51, 1999
[6] Hara et al., “Lipid extraction of tissue with low toxicity solvent”, Analytic Biochemistry, 90: 420-426, 1978
[7] Pickova et al., “Early embryonic cleavage of pattern, hatching success and egg-lipid fatty acid composition: Comparison of cod stocks”, Canadian Journal of Fisheries and Aquatic Science, 54: 2410-2416, 1997
[8] Appelqvist, “Rapid methods of lipid extractions and fatty acid methyl ester preparation for seed and leaf tissue with special remarks on preventing the accumulation of lipids contaminants” Royal Swedish Academy of Science, 28: 551-570, 1968
[9] Moazzami et al., “Sesame seed is a rich source of dietary lignans”, Journal of the American Oil Chemistry Society, 83: 719-723, 2006
[10] Kamal-Eldin, “Minor components of vegetable oils”, In: (Ed) Fereidoon Shahidi, Bailey's Industrial Oil and Fat Products, Sixth Edition, Vol VI, pp. 483-523, 2005
[11] Segeln, “Preparation of isolated rat liver cells”, Methods Cell Biol., 13: 29-83, 1976
[12] Dennevig and Berg, “Endocytosis of galactose-terminated glycoproteins by isolated liver cells of the rainbow trout (Salmo gairdneri)”, Comparative Biochemistry and Physiology B, 82: 683-688, 1985
[13] Folch et al., “A simple method for the isolation and purification of total lipids from animal tissues”, Journal of Biological Chemistry, 226: 497-509, 1957
[14] Narce et al., “Method for evaluating the bioconversion of radioactive polyunsaturated fatty acids by use of reversed-phase liquid chromatography”, Journal of Chromatography, 448: 249-264, 1988
[15] Fredriksson et al., “Fatty acid and carotenoid composition of egg yolk as an effect of microalgae addition to feed formula for laying hens”, Food Chemistry, 99: 530-537, 2006
[16] Christiansen et al., “The effect of (−)carnitine on the metabolism of palmitate in liver cells isolated from fasted and refed rats”, FEBS Lett., 62: 313-317, 1976
[17] Lowry et al., “Protein measurement with the folin phenol reagent”, Journal of Biological Chemistry, 193: 265-275, 1951
[18] Peterson, “A simplification of the protein assay method of Lowry et al. which is more generally applicable”, Anal Biochem, 83: 346-356, 1977
[19] Small et al., “A sensitive spectrophotometric assay for peroxisomal acyl-coa oxidase”, Biochemical Journal, 227: 205-210, 1985

1-33. (canceled)

34. A compound feed for aquaculture comprising:
a lignan represented by formula (I): 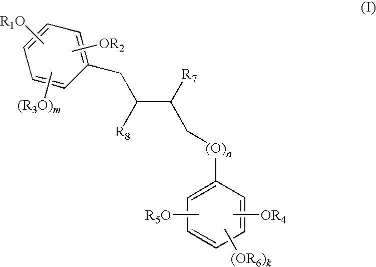
wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, m and n each independently represents 0 or 1, and R₇ and R₈ independently represents a methyl group or —CH₂—O— forming a tetrahydrofuran group with the lignan backbone;
a vegetable oil or vegetable oil mixture present in at least 20% by weight of said feed and having a total 18-carbon chain omega-3 fatty acid content of at least 30% by weight of said vegetable oil or vegetable oil mixture; and
an antioxidant.

35. The feed according to claim 34, wherein said lignan is a dioxabicyclo-[3.3.0]octane derivate represented by formula (II):

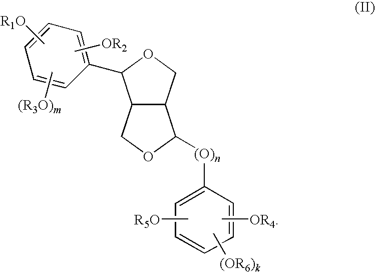

36. The feed according to claim 35, wherein said dioxabicyclo[3.3.0]octane derivate is selected from a group consisting of:
2,6-bis-(3,4-methylenedioxy phenyl)-cis-3,7-dioxabicyclo[3.3.0]octane;
2,6-bis-(3,4-methylenedioxy phenyl)-trans-3,7-dioxabicyclo[3.3.0]octane;
2-(3,4-methylenedioxy-6-hydroxy phenyl)-6-(3,4-methylenedioxy phenyl)-cis-3,7-dioxabicyclo[3.3.0]octane;
2-(3,4-methylenedioxy-6-hydroxy phenyl)-6-(3,4-methylenedioxy phenyl)-trans-3 dioxabicyclo[3.3.0]octane;
2-(3,4-methylenedioxy phenoxy)-6-(3,4-methylenedioxy phenyl)-cis-3,7-dioxabicyclo[3.3.0]octane;
2-(3-methoxy-4-hydroxy phenoxy)-6-(3,4-methylenedioxy phenyl)-cis-3,7-dioxabicyclo[3.3.0]octane;
2-(3-methoxy-4-hydroxy phenyl)-6-(3,4-methylenedioxy phenyl)-cis-3,7-dioxabicyclo[3.3.0]octane;
2,6-bis-(3-methoxy-4-hydroxy phenyl)-cis-3,7-dioxabicyclo[3.3.0]octane;
2-(3,4-methylenedioxy-6-methoxy phenyl)-6-(3,4-methylenedioxy phenyl)-cis-3,7-dioxabicyclo[3.3.0]octane;
2-(3,4-5-methylenedioxy phenyl)-6-(3,4-methylenedioxy-5-methoxy phenyl)-trans-3,7-dioxabicyclo[3.3.0]octane;
a glycoside of a dioxabicyclo[3.3.0]octane derivates of said group; and
a mixture of at least two dioxabicyclo[3.3.0]octane derivates of said group.

37. The feed according to claim 34, wherein said lignan is present in at least 0.5% by weight of said vegetable oil or vegetable oil mixture.

38. The feed according to claim 34, wherein said lignan is present in 0.1 to 10% by dry weight of said feed.

39. The feed according to claim 34, wherein said vegetable oil or vegetable oil mixture has a total 18-carbon chain omega-3 fatty acid content of at least 40% by weight of said vegetable oil or vegetable oil mixture.

40. The feed according to claim 34, wherein said vegetable oil is selected from a group consisting of linseed oil, flaxseed oil, perilla seed oil, soybean oil, rapeseed oil, hempseed oil, black current seed oil, Boraginaceae oil, a mixture of at least two vegetable oils of said group or a mixture of at least one vegetable oil of said group with another vegetable oil.

41. The feed according to claim 34, wherein said vegetable oil or vegetable oil mixture is present in 20 to 35% by weight of said feed.

42. The feed according to claim 34, wherein said antioxidant is a carotenoid.

43. The feed according to claim 42, wherein said carotenoid is selected from a group consisting of astaxanthin and cantaxanthin.

44. The feed according to claim 34, wherein said antioxidant is present in at least 5 mg/kg by dry weight of said feed.

45. The feed according to claim 34, wherein said vegetable oil or vegetable oil mixture has a polyunsaturated fatty acid content of at least 50% of a total fatty acid content of said vegetable oil or vegetable oil mixture.

46. The feed according to claim 34, wherein said 18-carbon chain omega-3 fatty acid is selected from a group consisting of α-linolenic acid and stearidonic acid.

47. A method of altering muscle fatty acid composition in an aquaculture species comprising feeding said aquaculture species with a compound feed for aquaculture comprising:
a lignan represented by formula (I): 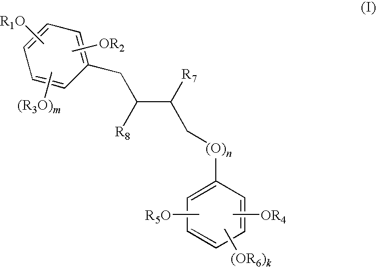
wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, in and n each independently represents 0 or 1, and R₇ and R₈ independently represents a methyl group or —CH₂—O— forming a tetrahydrofuran group with the lignan backbone;
a vegetable oil or vegetable oil mixture present in at least 20% by weight of said feed and having a total 18-carbon chain omega-3 fatty acid content of at least 30% by weight of said vegetable oil or vegetable oil mixture; and
an antioxidant.

48. The method according to claim 47, wherein said aquaculture species is a carnivorous aquaculture species.

49. The method according to claim 48, wherein said aquaculture species is selected from a group consisting of a carnivorous, cold water aquaculture species and a carnivorous, temperate water aquaculture species.

50. The method according to claim 47, wherein said aquaculture species is selected from a group consisting of salmonids, gadoids, perciformes and flat fishes.

51. A method of manufacturing a compound feed for aquaculture comprising mixing a lignan represented by formula (I): 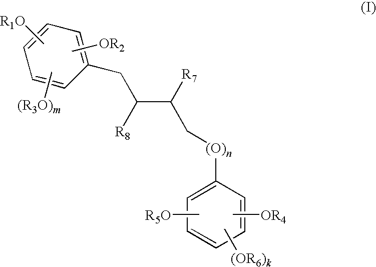
wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, m and n each independently represents 0 or 1, and R₇ and R₈ independently represents a methyl group or —CH₂—O— forming a tetrahydrofuran group with the lignan backbone, a vegetable oil or vegetable oil mixture present in at least 20% by weight of said feed and having a total 18-carbon chain omega-3 fatty acid content of at least 30% by weight of said oil or oil mixture, and an antioxidant.

52. A method of increasing a proportion of N-carbon chain omega-3 fatty acids in muscles of an aquaculture species, where N>18, comprising feeding said aquaculture species with a lignan represented by formula (I): 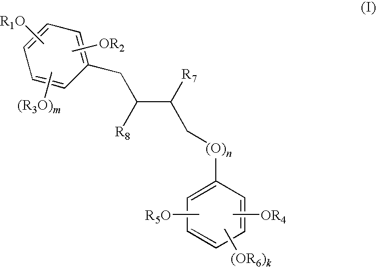
wherein R₁ to R₆ each independently represents an hydrogen atom, an alkyl group having 1 to 3 carbon atoms or a sugar moiety, or R₂ and R₂ and/or R₄ and R₅ together represent a methylene group or an ethylene group and k, m and n each independently represents 0 or 1, and R₇ and R₈ independently represents a methyl group or —CH₂—O— forming a tetrahydrofuran group with the lignan backbone.

53. The method according to claim 52, wherein said lignan is used for increasing a proportion of said N-carbon chain omega-3 fatty acids in white muscle of an aquaculture species.

54. The method according to claim 53, wherein said lignan is used for increasing a proportion of said N-carbon chain omega-3 fatty acids in at least one of white muscle phospholipids and triacylglycerol of said aquaculture species.

55. The method according to claim 52, wherein said N-carbon chain omega-3 fatty acids comprise at least one of eicosapentaenoic acid, docosapentaenoic acid and docosahexaenoic acid.

56. A method of increasing desaturation and elongation of at least one of stearidonic acid and α-linolenic acid in muscles of an aquaculture species comprising feeding said aquaculture species with a lignan represented by formula (I): 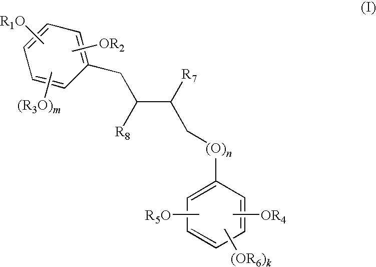
wherein R₁ to R₆ each independently represents an hydrogen atom, an alkyl group having 1 to 3 carbon atoms or a sugar moiety, or R₁ and R₂ and/or R₄ and R₅ together represent a methylene group or an ethylene group and k, m and n each independently represents 0 or 1, and R₇ and R₈ independently represents a methyl group or —CH₂—O— forming a tetrahydrofuran group with the lignan backbone.

57. The method according to claim 56, wherein said lignan is used for increasing desaturation and elongation of at least one of stearidonic acid and α-linolenic acid into at least one of eicosapentaenoic acid, docosapentaenoic acid and docosahexaenoic acid in said muscles of said aquaculture species.


SEQUENCE LISTING (22 sequences)

SEQ ID NO: 1 - DNA, 23 bp, Salmo salar
  ref <308>: DATABASE ACCESSION NUMBER: CA049789
  ref <309>: DATABASE ENTRY DATE: 2002-10-24
  ref <313>: RELEVANT RESIDUES IN SEQ ID NO: (156)..(178)
taacgcctgcctcttcacgttga

SEQ ID NO: 2 - DNA, 23 bp, Salmo salar
  ref <308>: DATABASE ACCESSION NUMBER: CA049789
  ref <309>: DATABASE ENTRY DATE: 2002-10-24
  ref <313>: RELEVANT RESIDUES IN SEQ ID NO: (245)..(267)
atgagggaccttgtagccagcaa

SEQ ID NO: 3 - DNA, 24 bp, Salmo salar
  ref <308>: DATABASE ACCESSION NUMBER: AF321836
  ref <309>: DATABASE ENTRY DATE: 2006-02-14
  ref <313>: RELEVANT RESIDUES IN SEQ ID NO: (121)..(144)
caccaccggccatctgatctacaa

SEQ ID NO: 4 - DNA, 24 bp, Salmo salar
  ref <308>: DATABASE ACCESSION NUMBER: AF321836
  ref <309>: DATABASE ENTRY DATE: 2006-02-14
  ref <313>: RELEVANT RESIDUES IN SEQ ID NO: (175)..(198)
tcagcagcctccttctcgaacttc

SEQ ID NO: 5 - DNA, 19 bp, Salmo salar
  ref <308>: DATABASE ACCESSION NUMBER: AJ416953
  ref <309>: DATABASE ENTRY DATE: 2006-02-03
  ref <313>: RELEVANT RESIDUES IN SEQ ID NO: (1018)..(1036)
ccagcaacccgtccttgtt

SEQ ID NO: 6 - DNA, 20 bp, Salmo salar
  ref <308>: DATABASE ACCESSION NUMBER: AJ416953
  ref <309>: DATABASE ENTRY DATE: 2006-02-03
  ref <313>: RELEVANT RESIDUES IN SEQ ID NO: (886)..(905)
gagacggtcagggagctcac

SEQ ID NO: 7 - DNA, 21 bp, Salmo salar
  ref <308>: DATABASE ACCESSION NUMBER: DQ294237
  ref <309>: DATABASE ENTRY DATE: 2006-09-27
  ref <313>: RELEVANT RESIDUES IN SEQ ID NO: (165)..(185)
cgttgaatttcatggcgaact

SEQ ID NO: 8 - DNA, 19 bp, Salmo salar
  ref <308>: DATABASE ACCESSION NUMBER: DQ294237
  ref <309>: DATABASE ENTRY DATE: 2007-09-27
  ref <313>: RELEVANT RESIDUES IN SEQ ID NO: (75)..(93)
tcctggtggcctacggatc

SEQ ID NO: 9 - DNA, 20 bp, Salmo salar
  ref <308>: DATABASE ACCESSION NUMBER: AJ292963
  ref <309>: DATABASE ENTRY DATE: 2006-01-11
  ref <313>: RELEVANT RESIDUES IN SEQ ID NO: (609)..(628)
cattgtcagcctgtccagac

SEQ ID NO: 10 - DNA, 20 bp, Salmo salar
  ref <308>: DATABASE ACCESSION NUMBER: AJ292963
  ref <309>: DATABASE ENTRY DATE: 2006-11-14
  ref <313>: RELEVANT RESIDUES IN SEQ ID NO: (867)..(878)
atgtgacattcccacaagca

SEQ ID NO: 11 - DNA, 24 bp, Oncorhynchus mykiss
  ref <308>: DATABASE ACCESSION NUMBER: DQ266043
  ref <309>: DATABASE ENTRY DATE: 2005-11-15
  ref <313>: RELEVANT RESIDUES IN SEQ ID NO: (316)..(339)
aactcagtgaagaggccaaacttg

SEQ ID NO: 12 - DNA, 16 bp, Oncorhynchus mykiss
  ref <308>: DATABASE ACCESSION NUMBER: DQ266043
  ref <309>: DATABASE ENTRY DATE: 2005-11-15
  ref <313>: RELEVANT RESIDUES IN SEQ ID NO: (136)..(151)
tgcggcggtgatgatg

SEQ ID NO: 13 - DNA, 21 bp, Oncorhynchus mykiss
ggatgaactccctgcatgtga

SEQ ID NO: 14 - DNA, 21 bp, Oncorhynchus mykiss
tgaggccaaagtactcgtcga

SEQ ID NO: 15 - DNA, 20 bp, Salmo salar
  ref <308>: DATABASE ACCESSION NUMBER: AF478472
  ref <309>: DATABASE ENTRY DATE: 2004-11-19
  ref <313>: RELEVANT RESIDUES IN SEQ ID NO: (305)..(324)
gagagctggcaccgacagag

SEQ ID NO: 16 - DNA, 20 bp, Salmo salar
  ref <308>: DATABASE ACCESSION NUMBER: AF478472
  ref <309>: DATABASE ENTRY DATE: 2004-11-19
  ref <313>: RELEVANT RESIDUES IN SEQ ID NO: (336)..(355)
gagctgcatttttcccatgg

SEQ ID NO: 17 - DNA, 20 bp, Salmo salar
  ref <308>: DATABASE ACCESSION NUMBER: AY458652
  ref <309>: DATABASE ENTRY DATE: 2004-11-19
  ref <313>: RELEVANT RESIDUES IN SEQ ID NO: (232)..(251)
agagcgtagctgacacagcg

SEQ ID NO: 18 - DNA, 20 bp, Salmo salar
  ref <308>: DATABASE ACCESSION NUMBER: AY458652
  ref <309>: DATABASE ENTRY DATE: 2004-11-19
  ref <313>: RELEVANT RESIDUES IN SEQ ID NO: (263)..(282)
tcctcggttctctctgctcc

SEQ ID NO: 19 - DNA, 21 bp, Salmo salar
  ref <308>: DATABASE ACCESSION NUMBER: DQ364432
  ref <309>: DATABASE ENTRY DATE: 2006-09-27
  ref <313>: RELEVANT RESIDUES IN SEQ ID NO: (1)..(21)
ccttcattgtacctctccgca

SEQ ID NO: 20 - DNA, 23 bp, Salmo salar
  ref <308>: DATABASE ACCESSION NUMBER: DQ364432
  ref <309>: DATABASE ENTRY DATE: 2006-09-27
  ref <313>: RELEVANT RESIDUES IN SEQ ID NO: (79)..(101)
catttcaacctcatcaaagccaa

SEQ ID NO: 21 - DNA, 22 bp, Salmo salar
  ref <308>: DATABASE ACCESSION NUMBER: AM230810
  ref <309>: DATABASE ENTRY DATE: 2006-03-02
  ref <313>: RELEVANT RESIDUES IN SEQ ID NO: (189)..(210)
gtaccagccccgatgccttcat

SEQ ID NO: 22 - DNA, 22 bp, Salmo salar
  ref <308>: DATABASE ACCESSION NUMBER: AM230810
  ref <309>: DATABASE ENTRY DATE: 2006-03-02
  ref <313>: RELEVANT RESIDUES IN SEQ ID NO: (290)..(311)
tctctgtgcgaccctctcggaa